(19) United States
(12) Patent Application Publication
(10) Pub. No.: US 2009/0130225 A1
(43) Pub. Date: May 21, 2009

(54) MACROLIDES DERIVATIVES AS ANTIBACTERIAL AGENTS

(76) Inventors: Anjan Chakrabarti, Gurgaon (IN); Biswajit Das, Gurgaon (IN); Jitendra Sambhaji Jadhav, Mahrashtra (IN); Dilip J. Upadhyay, Maharashtra (IN)

Correspondence Address: RANBAXY INC., 600 COLLEGE ROAD EAST, SUITE 2100, PRINCETON, NJ, 08540, US

(21) Appl. No.: 12/094,800

(22) PCT Filed: Nov. 21, 2006
(86) PCT No.: PCT/IB2006/054373

§ 371 Date: Oct. 23, 2008

(30) Foreign Application Priority Data
Nov. 23, 2005 (IN) 3135/DEL/2005

(51) Int. Cl. A61K 31/7048; C07H 17/08; A61K 36/28; A61K 36/54; A61K 33/06; A61K 33/24; A61K 33/30; A61P 3/04; A61P 1/00; C07D 403/04

(52) U.S. Cl. 424/617; 536/7.4; 514/29; 424/764; 424/737; 424/743; 424/682; 424/641; 544/331

(57) ABSTRACT

The present invention provides macrolide derivatives, which can be used as antibacterial agents. Compounds described herein can be used for treating or preventing conditions caused by or contributed to by gram-positive, gram-negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Propionibeacterium, Bacillus, Enterobactericeae or any combination thereof. Also provided are processes for preparing compounds described herein, pharmaceutical compositions thereof, and methods of treating bacterial infections.

### FIELD OF THE INVENTION

The present invention provides macrolide derivatives, which can be used as antibacterial agents. Compounds described herein can be used for treating or preventing conditions caused by or contributed to by gram-positive, gram-negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Propionibeacterium, Bacillus, Enterobactericeae or any combination thereof. Also provided are processes for preparing compounds described herein, pharmaceutical compositions thereof, and methods of treating bacterial infections.

### BACKGROUND OF THE INVENTION

First generation macrolides erythromycin A and early derivatives are characterized by bacteriostatic or bactericidal activity for most gram-positive bacteria, atypical pathogens, and many community-acquired respiratory infections and in patients with penicillin allergy. However, erythromycin A causes numerous drug-drug interactions, has relatively poor absorption, poor local tolerance, loses its antibacterial activity under acidic conditions by degradation and the degraded products are known to be responsible for undesired side effects (Itoh, Z et al., Am. J. Physiol, 1984, 247:688; Omura, S et al., J. Med. Chem., 1987, 30: 1943). Various erythromycin A derivatives have been prepared to overcome the acid instability and other problems associated with it.

Roxithromycin, clarithromycin and azithromycin were developed to address the limitation of erythromycin A. Both clarithromycin and azithromycin were found to be important drugs in the treatment and prophylaxis of atypical mycobacterial infections in patients with HIV.

Macrolides were found to be effective drugs in the treatment of many respiratory tract infections. However, increasing resistance among S. pneumoniae has prompted the search for new compounds that retain favorable safety profiles, retain a spectrum of activity and are confined to respiratory pathogens. Consequently, numerous investigators have prepared chemical derivatives of erythromycin A in an attempt to obtain analogs having modified or improved profiles of antibiotic activity. Ketolides exhibit greater efficacy and safety, have broader spectrum of activities, and are particularly effective against resistant pathogens; hence, ketolides have been developed as next generation macrolides.

U.S. Pat. No. 5,635,485 discloses erythromycin compounds that are reportedly useful in the treatment of bacterial infections in warm-blooded animals. U.S. Pat. No. 5,866,549 discloses novel semi-synthetic macrolides reportedly having antibacterial activity, as well as 6-O-substituted erythromycin ketolide derivatives and a method of treating bacterial infections. U.S. Pat. Nos. 6,458,771 and 6,399,582 and PCT Publication Nos. WO 00/62783 and WO 00/44761 disclose ketolide antibacterials that are reportedly useful in treating bacterial and protozoal infections and in treating other conditions involving gastric motility. U.S. Pat. No. 5,747,467 discloses erythromycin and novel antibacterial composition and a method of treating bacterial infection in warm-blooded animals. U.S. Pat. No. 6,433,151 discloses erythromycin derivatives and their use as medicament for treating infections caused by particular gram-positive bacteria, namely Haemophilus influenzae, and Morraxalla spp. U.S. Pat. No. 6,472,372 discloses 6-O-carbamoyl ketolide antibacterials and methods of treating bacterial infections. U.S. Patent Application Nos. 2002/0115621 and 2003/0013665 disclose macrolide compounds that are useful as antibacterial and antiprotozoal agents in mammals, including man, as well in fish and birds. U.S. Patent Application No. 2005/0153905 discloses novel antimicrobial ketolide compounds and U.S. Pat. No. 6,756,359 discloses C12 modified erythromycin macrolides and ketolides having antibacterial activity. Other ketolide compounds have also been reported. A. Denis and A. Bonnefoy, Drugs of the Future, 26(10):975-84 (2001), Champney W. S., et al., Current Microbiology, 42 :203-10 (2001).

However, there remains a need for macrolide derivatives, which can be used as antibacterial agents on a wide variety of gram-positive, gram-negative or anaerobic bacteria.

### SUMMARY OF THE INVENTION

Provided herein are macrolide derivatives, which can be used to treat or prevent bacterial infections, and processes for the synthesis of these compounds. Pharmaceutically acceptable salts, pharmaceutically acceptable solvates, enantiomers, diastereomers, polymorphs of these compounds having same type of activity are also provided. Pharmaceutical compositions containing one or more compounds described herein together with one or more pharmaceutically acceptable carriers, excipients, diluents or mixture thereof, which can be used to treat bacterial infections.

Thus, in one aspect, provided herein are compounds having the structure of Formula I,

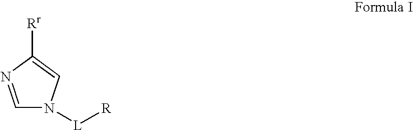

pharmaceutically acceptable salts, pharmaceutically acceptable solvates, enantiomers, diastereomers or polymorphs thereof, wherein:
R^{r} can be

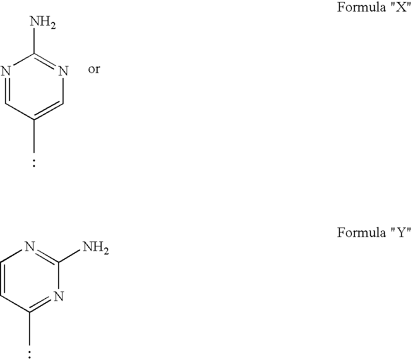

NH₂ can be optionally substituted;
   substitutents can be alkyl or can form a heterocyclic ring together with nitrogen atom;
L can be a linker;
R can be

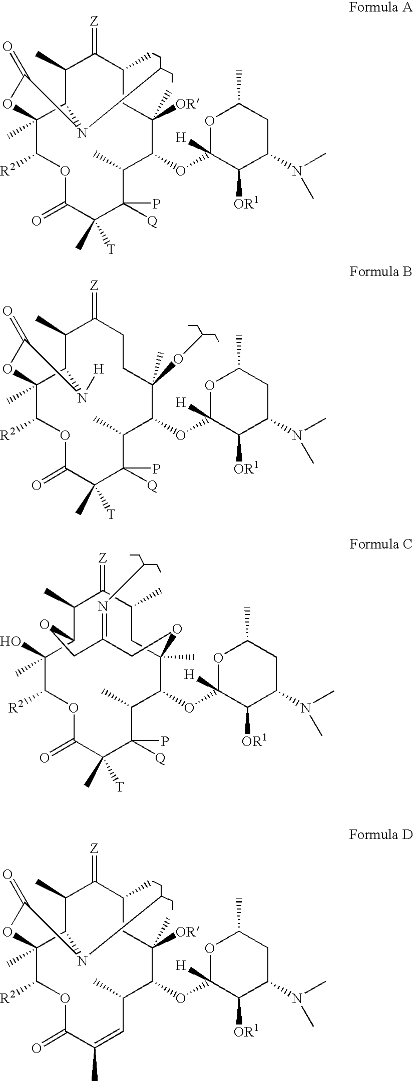

R¹ can be hydrogen or hydroxy protecting group;
   R² can be alkyl, alkenyl or alkynyl;
   R′ can be alkyl or —(CH₂)ᵣ—U;
      r can be an integer of from 1 to 4;
      U can be alkenyl or alkynyl;
   Z can be oxygen, sulfur or NAc, NOR⁴;
      R⁴ can be hydrogen, alkyl or arylalkyl;
   T can be hydrogen, halogen, cyano or alkyl;
P and Q can be independently hydrogen, hydroxy, OC(=Z′)VR³ or

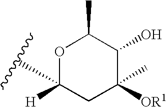

P and Q together can form oxo or thioxo group;
   Z′ can be oxygen or sulfur;
V can be —W(CH₂)ₖ—;
   k can be an integer of from 1 to 6;
   W can be no atom, —NR⁵— or oxygen;
      R⁵ can be hydrogen or alkyl;
   alkylene chain of —W(CH₂)ₖ— can be optionally substituted with alkyl, hydroxy or alkoxy;
R³ can be alkyl, aryl or heterocycle.

Compounds of Formula I may involve one or more of the following embodiments. For example, provided herein are compounds having the structure of Formula II,

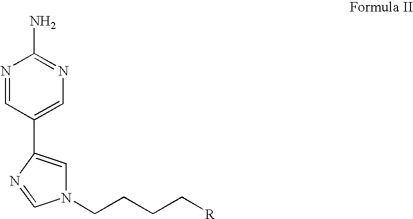

wherein NH₂ can be optionally substituted and R has the same meaning as defined earlier.

In another embodiment, provided herein are compounds of Formula III,

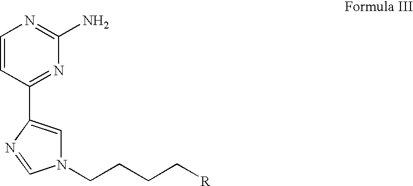

wherein NH₂ can be optionally substituted and R has the same meaning as defined earlier.

In another aspect, provided herein are compounds selected from:

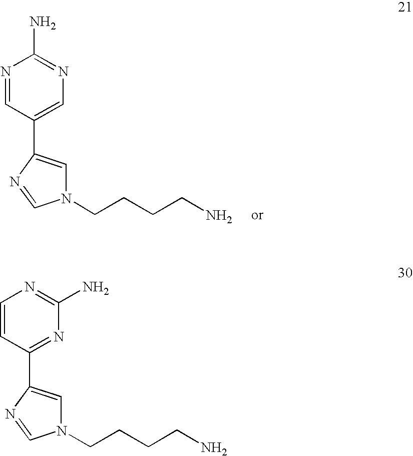

In another aspect, provided herein are compounds selected from:
R^{r} is Formula “X”; L is (CH₂)₄; R is Formula A [P and Q together form oxo group, R¹=H, R²=Et, Z=O, R′=Me, T=H],
R^{r} is Formula “Y”; L is (CH₂)₄; R is Formula A [P and Q together form oxo group, R¹H, R²=Et, Z=O, R′=Me, T=H],
R^{r} is Formula “X”; L is (CH₂)₄; R is Formula A [P═H, Q=O-(2-pyridyl acetyl), R¹=H, R²=Et, Z=O, R′=Me, T=H],
R^{r} is Formula “X”; L is (CH₂)₄; R is Formula A [P═H, Q=O-(3-pyridyl acetyl), R¹=H, R²=Et, Z=O, R′=Me, T=H],
R^{r} is Formula “X”; L is (CH₂)₄; R is Formula A [P═H, Q=O-(2-fluorophenyl acetyl), R¹=H, R²=Et, Z=O, R′=Me, T=H],
R^{r} is Formula “X”; L is (CH₂)₄; R is Formula A [P═H, Q=O-(3-fluorophenyl acetyl), R¹=H, R²=Et, Z=O, R′=Me, T=H],
R^{r} is Formula “Y”; L is (CH₂)₄; R is Formula A [P═H, Q=O-(3-fluorophenyl acetyl), R¹=H, R²=Et, Z=O, R′=Me, T=H],
R^{r} is Formula “Y”; L is (CH₂)₄; R is Formula A [P═H, Q=O-(2-fluorophenyl acetyl), R¹=H, R²=Et, Z=O, R′=Me, T=H],
R^{r} is Formula “Y”; L is (CH₂)₄; R is Formula A [P═H, Q=O-(3-pyridyl acetyl), R¹=H, R²=Et, Z=O, R′=Me, T=H],
R^{r} is Formula “Y”; L is (CH₂)₄; R is Formula A [P═H, Q=O-(2-pyridyl acetyl), R¹=H, R²=Et, Z=O, R′=Me, T=H],
R^{r} is Formula “X”; L is (CH₂)₄; R is Formula A [P and Q together form oxo group, R¹=H, R²=Et, Z=O, R′=Me, T=F],
R^{r} is Formula “X”; L is (CH₂)₄; R is Formula A [P═H, Q=H, R¹=H, R²=Et, Z=O, R′=Me, T=H],
R^{r} is Formula “X”; L is (CH₂)₄; R is Formula D [R¹=H, R²=Et, Z=O, R′=Me] or

pharmaceutically acceptable salts, pharmaceutically acceptable solvates, enantiomers, diastereomers or polymorphs thereof.

In another aspect, provided herein are pharmaceutical compositions comprising therapeutically effective amounts of one or more compounds of compounds disclosed herein together with one or more pharmaceutically acceptable carriers, excipients, or diluents.

In another aspect, provided herein are methods for treating or preventing conditions caused by or contributed to by bacterial infections comprising administering to a mammal in need thereof therapeutically effective amounts of one or more compounds of compounds disclosed herein.

The methods may include one or more of the following embodiments. For example, the condition can be selected from community acquired pneumonia, upper or lower respiratory tract infections, skin or soft tissue infections, hospital acquired lung infections, hospital acquired bone or joint infections, mastitis, catheter infection, foreign body, prosthesis infections or peptic ulcer disease. In another embodiment, the bacterial infection can be caused by gram-positive, gram-negative or anaerobic bacteria.

In yet another embodiment, the gram-positive, gram-negative or anaerobic bacteria can be selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Propionibacterium, Bacillus or Enterobactericeae. In a preferred embodiment, the bacterium is cocci. In another preferred embodiment, the cocci are drug resistant.

In yet another aspect, provided herein are methods for treating or preventing acne vulgaris and inflammatory conditions thereof comprising administering to a mammal in need thereof therapeutically effective amounts of one or more compounds of Formula I in combination with one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid, and other therapeutic agents, which can be used to treat acne or condition the skin conditions.

In another aspect, provided herein are processes for preparing compounds of Formula I.

The following definitions apply to terms as used herein:

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulfinyl, sulfonyl group or —NRₐ—, wherein Rₐ can be hydrogen, alkyl, alkenyl, alkynyl, cycloalkyl or aryl. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further (referred herein as “substituted alkyl”) with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)Rₖₚ, —NRₚR_{q}, —C(═O)NRₚR_{q}, —NHC(═O)NRₚR_{q}, —C(═O)heteroaryl, C(═O)heterocyclyl, —O—C(═O)NRₚR_{q} {wherein Rₚ and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, hydroxyamino, alkoxyamino or S(O)ₘR₆₆ (wherein m is an integer from 0-2 and R₆₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NRₚR_{q}, —C(═O)NRₚR_{q}, —OC(═O)NRₚR_{q}, —NHC(═O)NR_{fp}R_{q} (wherein Rₚ and R_{q} are the same as defined earlier), hydroxy, alkoxy, halogen, CF₃, cyano, and S(O)ₘR₆₆ (wherein m is an integer from 0-2 and R₆₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)ORₚ (wherein Rₚ is the same as defined earlier), S(O)ₘR₆₆ (wherein m is an integer from 0-2 and R₆₆ is as defined earlier), or —C(═O)NRₚR_{q} (wherein Rₚ and R_{q} are as defined earlier)}. Unless otherwise constrained by the definition, all substituents may be substituted further by 1-3 substituents selected from alkyl, carboxy, carboxyalkyl, —NRₚR_{q}, —C(═O)NRₚR_{q}, —O—C(═O)NRₚR_{q} (wherein Rₚ and R_{q} are the same as defined earlier), hydroxy, alkoxy, halogen, CF₃, cyano, and S(O)ₘR₆₆ (wherein m is an integer from 0-2 and R₆₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. It can be optionally interrupted by atom(s) or group(s) independently chosen from oxygen, sulfur, phenylene, sulfinyl, sulfonyl and —NRₐ—, wherein Rₐ can be hydrogen, alkyl, alkenyl, alkynyl, cycloalkyl or aryl. In the event that alkenyl is attached to a heteroatom, the double bond cannot be alpha to the heteroatom. Alkenyl groups may be substituted further (referred to herein as “substituted alkenyl”) with one or more substituents selected from alkyl, alkenyl, alkynyl, alkoxy, cycloalkyl, cycloalkenyl, acyl, acylamino, acyloxy, —NHC(═O)Rₚ, —NRₚR_{q}, —C(═O)NRₚR_{q}, NHC(═O)NRₚR_{q}, —O—C(═O)NRₚR_{q} (wherein Rₚ and R_{q} are the same as defined earlier), alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclylalkyl, heteroarylalkyl, aminosulfonyl, aminocarbonylamino, alkoxyamino, hydroxyamino, alkoxyamino, nitro, or SO₂R₆₆ (wherein R₆₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NRₚR_{q}, —C(═O)NRₚR_{q}, —O—C(═O)NRₚR_{q} (wherein Rₚ and R_{q} are the same as defined earlier) and —SO₂R₆₆ (where R₆₆ is same as defined earlier). Groups such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. It can be optionally interrupted by atom(s) or group(s) independently chosen from oxygen, sulfur, phenylene, sulfinyl, sulfonyl and —NRₐ—, where Rₐ can be hydrogen, alkyl, alkenyl, alkynyl, cycloalkyl or aryl. In the event that alkynyl is attached to a heteroatom, the triple bond cannot be alpha to the heteroatom. Alkynyl groups may be substituted further (referred to herein as “substituted alkynyl”) with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)Rₚ, —NRₚR_{q}, —NHC(═O)NRₚR_{q}, —C(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NRₚR_{q}, —C(═O)NRₚR_{q}, —NHC(═O)NRₚR_{q}, —C(═O)NRₚR_{q} (wherein Rₚ and R_{q} are the same as defined earlier), cyano, or S(O)ₘR₆₆ (wherein m is an integer from 0-2 and R₆₆ is same as defined earlier). Groups such as ethynyl, (—C≡CH), propargyl (or propynyl, —CH₂C≡CH), and the like exemplify this term.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NRₚR_{q}, —NHC(═O)NRₚR_{q}, —NHC(═O)Rₚ, —C(═O)NRₚR_{q}, —O—C(═O)NRₚR_{q} (wherein Rₚ and R_{q} are the same as defined earlier), nitro, heterocyclyl, heteroaryl, heterocyclylalkyl, heteroarylalkyl, or S(O)ₘR₆₆ (wherein m is an integer from 0-2 and R₆₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NRₚR_{q}, —C(═O)NRₚR_{q}, —NHC(═O)NRₚR_{q}, —O—C(═O)NRₚR_{q} (wherein Rₚ and R_{q} are the same as defined earlier), cyano or S(O)ₘR⁶⁶ (wherein m is an integer from 0-2 and R⁶⁶ is same as defined earlier). As used herein the term “halogen or halo” refers to fluorine, chlorine, bromine or iodine.

As used herein the term “hydroxyl protected” includes, but is not limited to, acyl, aroyl, alkyl, aryl, butyldiphenylsilyl, methoxymethyl and methylthiomethyl, and the like.

As used herein the term “thiol” refers to the group —SH.

The term “alkoxy” denotes the group O-alkyl or O-cycloalkyl, wherein alkyl and cycloalkyl are the same as defined above. Examples of alkoxy include, but are not limited to, methoxy, ethoxy, cyclopentoxy, and the like.

As used herein the term “thioalkyl” refers to —SR⁶, wherein R⁶ is alkyl or cycloalkyl.

As used herein the term “haloalkyl” refers to alkyl of which one or more hydrogen(s) is/are replaced by halogen.

The term “aryl” herein refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₛ (wherein Rₛ is hydrogen, alkyl, alkenyl, cycloalkyl, aralkyl, heterocyclylalkyl, heteroarylalkyl), NHC(═O)Rₚ, —NRₚR_{q}, C(═O)NRₚR_{q}, —NHC(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined above. Examples of aralkyl include, but are not limited to, benzyl, napthylmethyl, phenethyl and phenylpropyl, and the like.

The terms “heterocycle” or “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, alkoxy, cycloalkyl, acyl, amino, optionally substituted aryl, alkoxy, alkaryl, cyano, nitro, oxo, carboxy, guanidine, haloalkyl, optionally substituted heterocyclyl, optionally substituted heterocyclylalkyl, heteroaryl, —CORₚ, —O—C(═O)Rₚ, —O—C(═O)ORₚ, C(═O)NRₚR_{q}, S(O)ₘR₆₆, —O—C(═O)NRₚR_{q}, nitro, NHC(═O)NRₚR_{q}, —NRₚR_{q} (wherein m, R₆₆, Rₚ and R_{q} are as defined earlier), —NHCORₚ, —NHSO₂Rₚ, and —SO₂NHRₚ, mercapto or thioalkyl.

Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocycles include, but not limited to, azabicyclohexyl, azetidinyl, benzoimidazolyl, 1,4-benzodioxanyl, 1,3-benzodioxolyl, benzoxazolyl, benzothiazolyl, benzothiazinyl, benzotriazolyl, benzoxazinyl, carbaxolyl, dihydrobenzofuryl, dihydroimidazolyl, dihydropyranyl, dihydrofuranyl, dihydroindolyl, dihydroisoxazolyl, dihydropyridinyl, dioxanyl, dioxolanyl, furyl, homopiperidinyl, imidazolyl, imidazolinyl, imidazolidinyl, imidazopyridinyl, indolinyl, indolyl, isoindole 1,3-dione, isoquinolinyl, isothiazolidinyl, isothiazolyl, isoxazolidinyl, isoxazolyl, morpholinyl, napthyridinyl, oxazolidinyl, oxazolyl, phenoxazinyl, phenothiazinyl, piperazinyl, piperidinyl, purinyl, pyrazinyl, pyrazolinyl, pyrazolyl, pyridinyl, pyridyl, pyrimidinyl, pyrrolidinyl, pyrrolinyl, pyrrolyl, pyrrolopyridinyl, quinolinyl, tetrahydrofuranyl, tetrahydropyranyl, tetrazolyl, thiazolidinyl and thiazolyl, and thienyl and the like.

As used herein the term “(heterocyclyl)alkyl” refers to heterocycle which is bonded to an alkylene chain, wherein heterocyclyl and alkyl are the same as defined above. Examples of heterocycle alkyl include, but are not limited to, isothiazolidinyl ethyl, isothiazolyl propyl, pyrazinyl methyl, pyrazolinyl propyl and pyridyl butyl, pyridyl methyl and the like.

As used herein the term “polymorphs” refers to all crystalline forms and amorphous forms of the compounds described herein. In addition, some of the compounds described herein may form solvates with water (i.e., hydrate, hemihydrate or sesquihydrate) or common organic solvents. Such solvates are also encompassed within the scope of this invention.

As used herein the term “Linker” refers to groups selected from alkenyl, -G(CH₂)_{q}J-, —CR⁹R¹⁰, —NR⁹— and —SO₂, wherein q can be an integer of from 2 to 6; G can be no atom, —O—, —CO, —CS or —SO₂; R⁹ and R¹⁰ can independently be hydrogen or alkyl; and J can be no atom, —CR⁹R¹⁰ or N(R¹²)(CH₂)ₘ, wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle; (CH₂)_{q} (when G=J=no atom) group can be optionally interrupted by groups independently chosen from oxygen, sulfur, alkenyl, or —NR⁷—, where R⁷ can be hydrogen, alkyl, cycloalkyl, alkenyl, heterocyclyl, (heterocyclyl)alkyl, alkynyl, aryl or aralkyl one of the hydrogen atom of (CH₂)_{q} can be optionally replaced by alkyl, hydroxy or alkoxy.

The phrase “pharmaceutically acceptable salts” denotes salts of the free base, which possess the desired pharmacological activity of the free base and which are neither biologically nor otherwise undesirable. Suitable pharmaceutically acceptable salts may be prepared from an inorganic or organic acid. Example of such inorganic acids include, but not limited to, hydrochloric, hydrobromic, hydroiodic, nitrous (nitrite salt), carbonic, sulfuric, phosphoric acid and like. Appropriate organic acids include, but not limited to, aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid and the like.

The term pharmaceutically acceptable carriers” is intended to include non-toxic, inert solid, semi-solid or liquid filler, diluent, encapsulating material or formulation auxiliary of any type.

The compounds of present invention include stereoisomers. The term “stereoisomer” refers to compounds, which have identical chemical composition, but differ with regard to arrangement of the atoms and the groups in space. These include enantiomers, diastereomers, geometrical isomers, atropisomer and comformational isomers. Geometric isomers may occur when a compound contains a double bond or some other feature that gives the molecule a certain amount of structural rigidity. An enantiomer is a stereoisomer of a reference molecule that is the nonsuperimposable mirror image of the reference molecule. A diastereomer is a stereoisomer of a reference molecule that has a shape that is not the mirror image of the reference molecule. An atropisomer is a conformation of a reference compound that converts to the reference compound only slowly on the NMR or laboratory time scale. Conformational isomers (or conformers or rotational isomers or rotamers) are stereoisomers produced by rotation about a bonds, and are often rapidly interconverting at room temperature. Racemic mixtures are also encompassed within the scope of this invention.

### DETAILED DESCRIPTION OF THE INVENTION

Compounds described herein may be prepared by techniques well known to one of ordinary skill in the art. In addition, compounds described herein may also be prepared by the following reaction sequences as depicted in Schemes I, II, Ia, IIb, IIc, III and IV below.

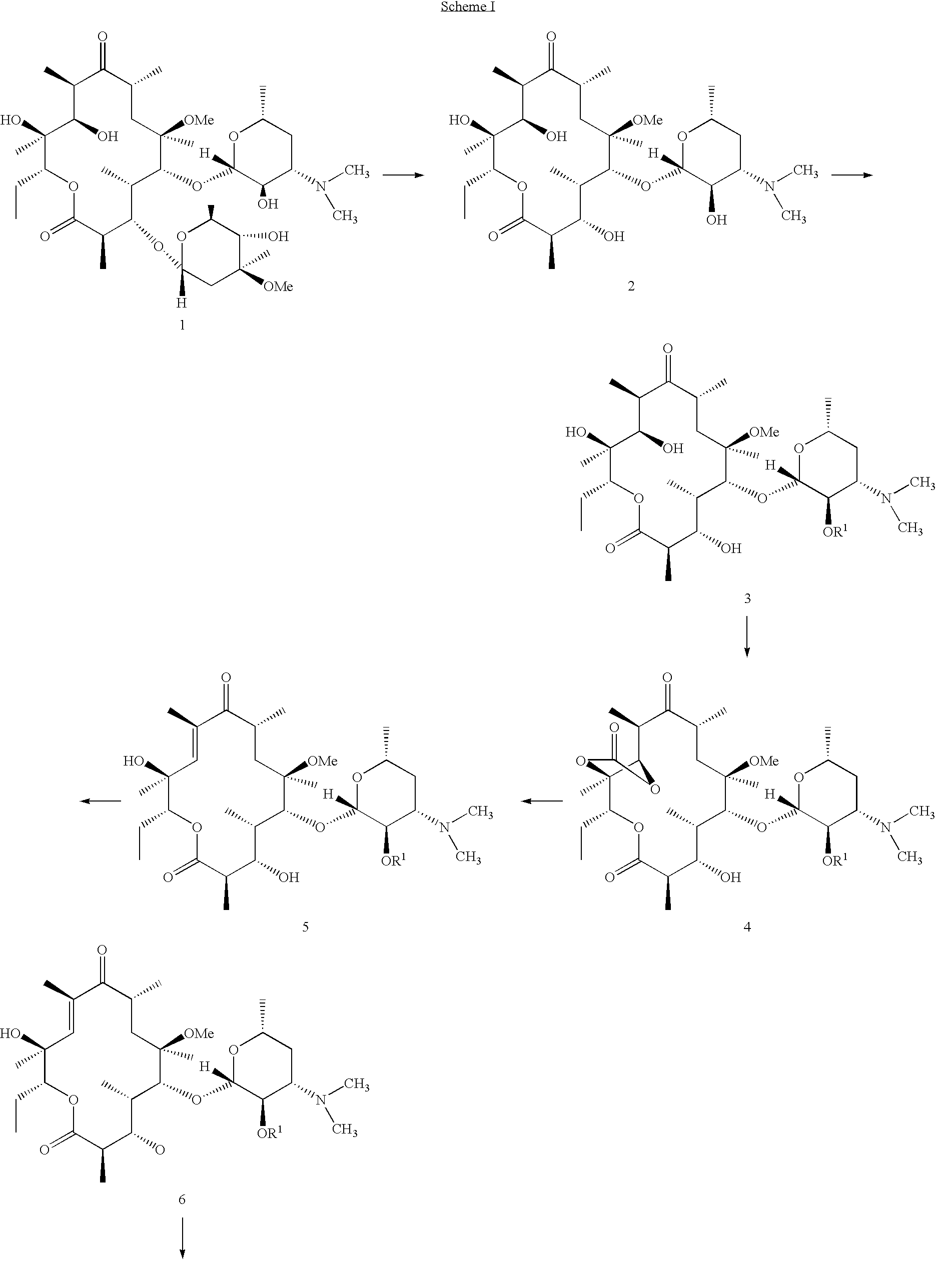

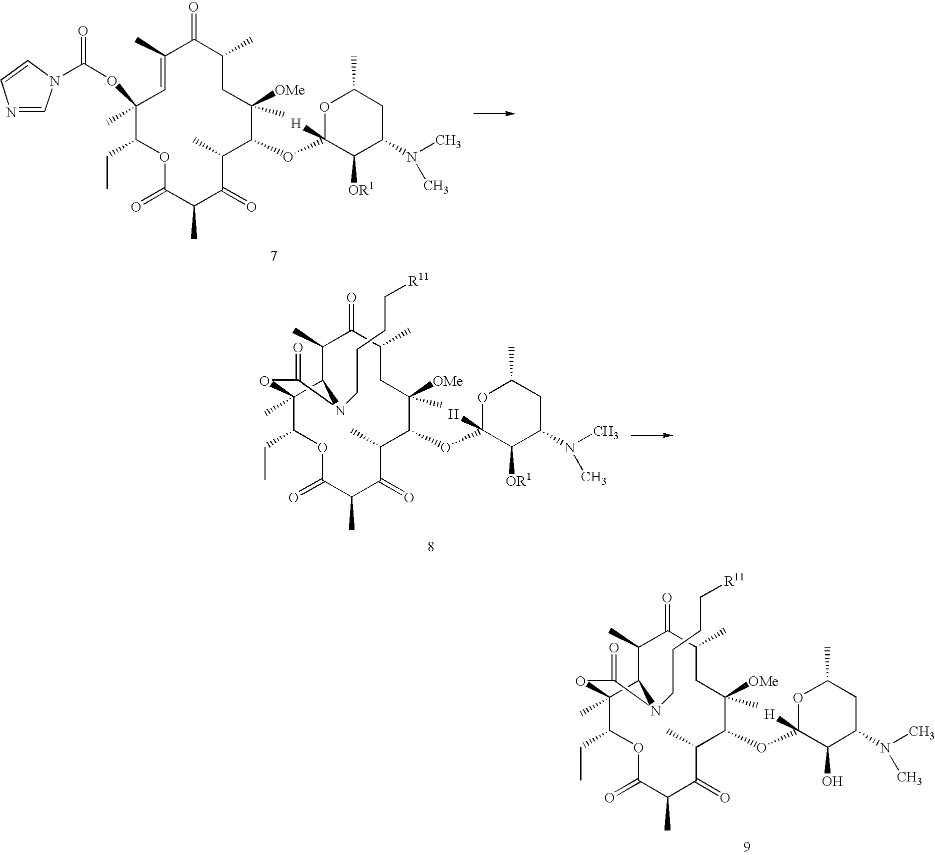

Compounds of Formula 9 can be prepared according to Scheme I. Thus, clarithromycin of Formula 1 can be hydrolyzed to form a compound of Formula 2. The compound of Formula 2 is protected with a reagent of Formula R¹₂O or R¹X (wherein X is halogen) to form compounds of Formula 3 (wherein R¹ is hydroxy protecting group, for example, COPh, tetrahydropyranyl, trialkylsilylethers and the like). The compound of formula 3 is reacted with a carbonylating reagent to form a compound of Formula 4. The compound of Formula 4 is reacted with an organic base, for example, tetramethyl guanidine, pyridine or trimethylamine to form a compound of Formula 5. The compound of Formula 5 is oxidized to form a compound of Formula 6. The compound of Formula 6 is reacted with N,N′-carbonyldiimidazole to form a compound of Formula 7. The compound of Formula 7 is reacted with a compound of Formula R¹¹ (CH₂)₄NH₂ to form a compound of Formula 8 (wherein R¹¹ is

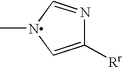

and R^{r} is the same as defined earlier). The compound of Formula 8 is deprotected to form a compound of Formula 9. The compound of Formula 9 is further converted into its salt by following the conventional method well known in the prior art.

The hydrolysis of clarithromycin of Formula 1 to form a compound of Formula 2 can be carried out in the presence of one or more acids, for example, inorganic acids (e.g., hydrochloric acid or sulphuric acid), organic acids (e.g., trifluoro acetic acid or dichloroacetic acid) or mixture thereof.

The protection of a compound of Formula 2 with a reagent of Formula R¹₂O or R¹X (wherein X is halogen) to form a compound of Formula 3 can be carried out in one or more solvents, for example, chlorinated solvents (e.g., dichloromethane, dichloroethane, chloroform or carbon tetrachloride), aprotic polar solvents (e.g., dimethylformamide, dimethylsulfoxide), nitriles (e.g., acetonitrile or propionitrile), acetates (e.g., ethyl acetate or methyl acetate) or mixture thereof. In the presence of one or more organic bases, for example, triethylamine, diisopropylethylamine, pyridine, tributylamine, 4-(N-dimethylamino)pyridine or mixture thereof.

The reaction of a compound of Formula 3 with a carbonylating reagent, for example, phosgene, triphosgene, N,N′-carbonyldiimidazole, ethyl chloroformate, ethyl trichloroacetate, o-phenylchloroformate or ethylene carbonate to give a compound of Formula 4 can be carried out in one or more chlorinated solvents, for example, chloroform, dichloromethane, carbon tetrachloride, dichloroethane or polar aprotic solvent (acetone, tetrahydrofuran) or mixture thereof. In the presence of one or more organic bases, for example, triethylamine, diisopropyl ethylamine, pyridine, tributylamine, 4-(N-dimethylamino)pyridine, sodium carbonate or mixture thereof.

The reaction of a compound of Formula 4 with an organic base, for example, tetramethyl guanidine, pyridine or trimethylamine to form a compound of Formula 5 can be carried out in one or more solvents, for example, aprotic polar solvents (e.g., dimethylformamide or dimethylsulfoxide), nitriles (e.g., acetonitrile or propionitrile), ethers (e.g., diethyl ether or tetrahydrofuran) or mixture thereof.

The oxidation of a compound of Formula 5 to give a compound of Formula 6 can be carried out with one or more oxidizing agent, for example, Dess-Martin periodinane, N-chloro succinimide, pyridinium chlorochromate, Swern Oxidation reagent (oxalyl chloride and dimethylsulfoxide), Pfitzner-Moffatt Oxidation reagent (dicyclohexylcarbodiimide and dimethylsulfoxide), Jones Oxidation reagent (chromic acid, aqueous sulfuric acid and acetone), pyridinium dichromate or 1-ethyl-3-(3-dimethylaminopropyl)carbodiimide hydrochloride. N-Chlorosuccinamide can be used in combination with dimethyl sulphide and 1-ethyl-3-(3-dimethylaminopropyl)carbodiimide hydrochloride can be used in combination with dimethylsulfoxide. The oxidation can also be carried out in one or more solvents, for example, chlorinated solvents (e.g., chloroform, dichloromethane, carbon tetrachloride or dichloroethane), aprotic polar solvents (e.g., dimethylsulfoxide or dimethylformamide), acetates (e.g., methyl acetate or ethyl acetate) or mixture thereof.

The reaction of a compound of Formula 6 with N,N′-carbonyldiimidazole to form a compound of Formula 7 can be carried out in one or more solvents, for example, ethers (e.g., tetrahydrofuran or diethyl ether), aprotic polar solvents (e.g., dimethylformamide or dimethylsulphoxide), nitriles (e.g., acetonitrile or propionitrile) or mixture thereof. In the presence of one or more bases, for example, alkali metal bases (e.g., sodium hydrogen carbonate, sodium acetate, sodium thiosulphate, sodium carbonate, potassium carbonate, cesium carbonate or sodium hydride), alkaline earth metal bases (e.g., calcium carbonate, or calcium hydroxide), triethylamine or mixture thereof.

The reaction of a compound of Formula 7 with a compound of Formula R¹¹(CH₂)₄NH₂ to form a compound of Formula 8 can be carried out in one or more solvents, for example, aprotic polar solvents (e.g., dimethylformamide or dimethylsulphoxide, dimethoxyethane, or tetrahydrofuran), protic polar solvents (methanol, ethanol, propanol or water), nitriles (e.g., acetonitrile or propionitrile) or mixture thereof.

The deprotection of a compound of Formula 8 to give a compound of Formula 9 can be carried out in one or more protic polar solvents, for example, methanol, ethanol, propanol, isopropanol, butanol, water or mixture thereof.

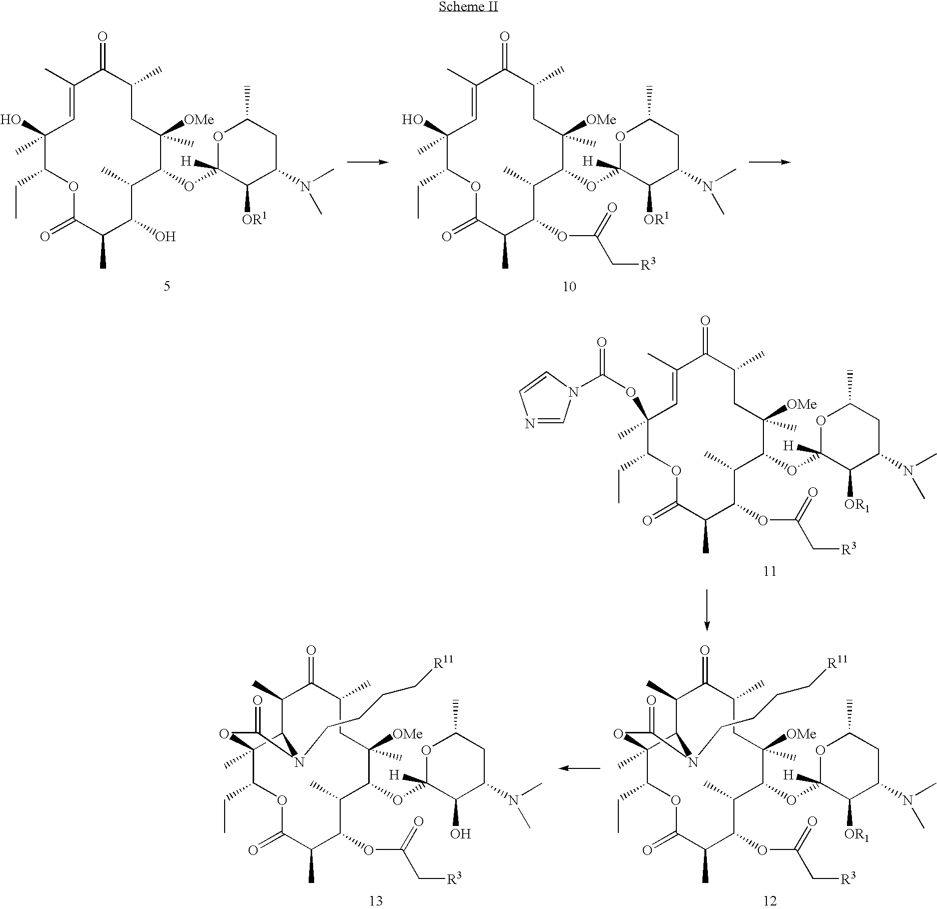

Compounds of Formula 13 can be prepared according to Scheme II. Thus, the compound of Formula 5 can be acylated with a reagent of Formula R³CH₂COOH, (R³CH₂CO)₂O, R³CH₂COX or R³CH₂COOL¹ (wherein L¹ is leaving group such as pivaloyl, p-toleuensulfonyl, isobutoxycarbonyl, ethoxycarbonyl or isopropoxycarbonyl) to form compounds of Formula 10 (wherein R¹ and R³ are same as defined earlier). The compound of Formula 10 can be reacted with N,N′-carbonyl diimidazole to form compounds of Formula 11. The compound of Formula 11 can be reacted with a compound of Formula R¹¹(CH₂)₄NH₂ to form compounds of Formula 12. The compound of Formula 12 can be deprotected to form compounds of Formula 13. The compound of Formula 13 can further be converted into its salt by following the conventional method well known in the prior art.

The reaction of a compound of Formula 5 with a acylating reagent of Formula R³CH₂COOH, (R³CH₂CO)₂O, R³CH₂COX or R³CH₂COOL¹ to form a compound of Formula 10 can be carried out in one or more solvents, for example, chlorinated solvents (e.g., chloroform, dichloromethane, dichloroethane or carbon tetrachloride), aprotic polar solvents (e.g., dimethylformamide or dimethylsulfoxide), ketones (e.g., acetone or ethyl methyl ketone), acetates (e.g., ethyl acetate or methyl acetate), ethers (e.g., tetrahydrofuran or diethyl ether) or mixture thereof. In the presence of one or more bases, for example, inorganic bases (e.g., sodium bicarbonate, sodium hydride or potassium carbonate), organic bases (e.g., triethylamine, pyridine, tributylamine or 4-N-dimethylaminopyridine) or mixture thereof with one or more activating agents, for example, dicyclohexylcarbodiimide, 1-ethyl-3-(3-dimethylaminopropyl)carbodiimide hydrochloride.

The reaction of a compound of Formula 10 with N,N′-carbonyldiimidazole to form a compound of Formula 11 can be carried out under similar conditions as that of conversion of a compound of Formula 6 to give a compound of Formula 7.

The reaction of a compound of Formula 11 with a compound of Formula R¹¹(CH₂)₄NH₂ to form a compound of Formula 12 can be carried out under similar conditions as that of conversion of a compound of Formula 7 to give a compound of Formula 8.

The deprotection of a compound of Formula 12 to give a compound of Formula 13 can be carried out similarly, as that of deprotection of a compound of Formula 8 to give a compound of Formula 9.

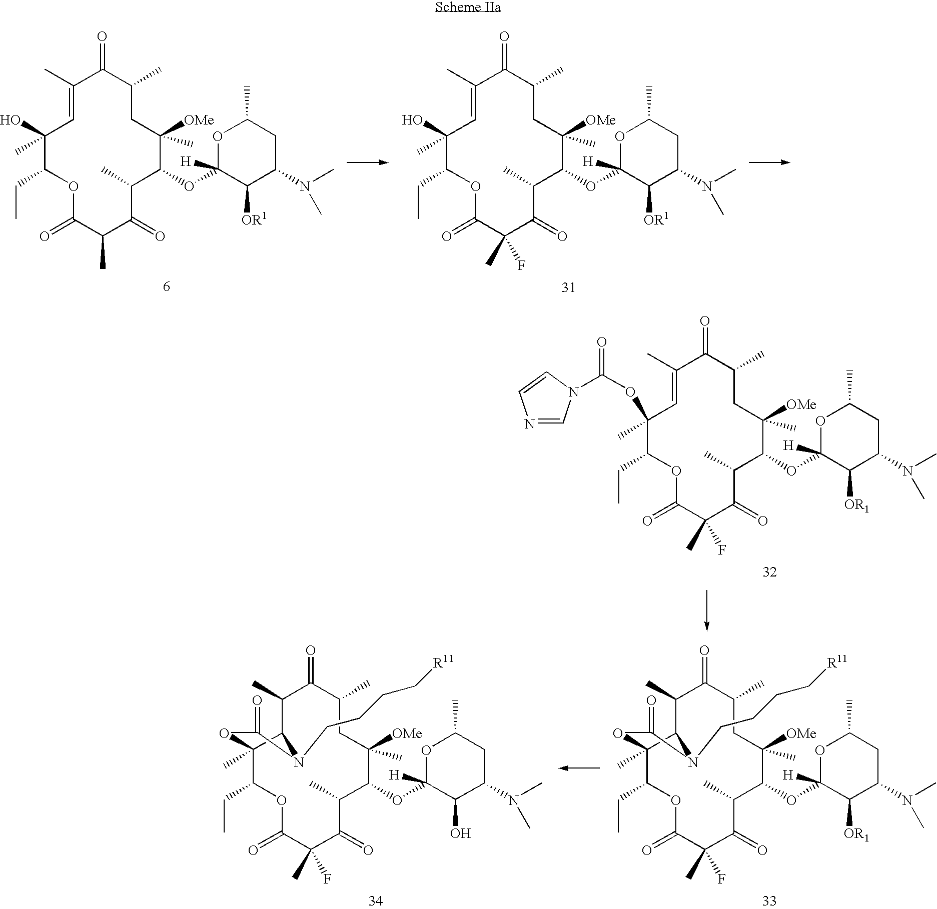

Compound of Formula 34 can be prepared according to Scheme IIa. Thus, reacting compound of Formula 6 with fluorinating agent gives a compound of Formula 31, which is reacted with N,N′-carbonyldiimidazole to form a compound of Formula 32. The compound of Formula 32 is further reacted with a compound of Formula R¹¹(CH₂)₄NH₂ to give a compound of Formula 33, which is then deprotected to give a compound of Formula 34. The compound of Formula 34 can further be converted into its salt by following the conventional method well known in the prior art.

The fluorination of a compound of Formula 6 to form a compound of Formula 31 can be carried out with one or more fluorinating agents, for example, N-Fluorobenzenesulphonimide, selectfluor or mixtures thereof, in one or more organic solvents, for example, tetrahydrofuran, dimethylformamide, dimethylsulfoxide or mixtures thereof in the presence of one or more base, for example, potassium tert-butoxide, potassium carbonate, sodium hydride, sodium acetate, sodium thiosulfate, sodium tert-butoxide, lithium diisopropylamide, sodium methoxide, sodium ethoxide, potassium methoxide, potassium ethoxide, sodium isopropoxide, potassium isopropoxide, lithium carbonate or mixtures thereof. The fluorination reactions can also be carried out by procedures disclosed in G. Sankar Lal and Syvret R. G., Chem. Rev., 96:1737-1755 (1996).

The reaction of compound of Formula 31 with N,N′-carbonyldiimidazole to form a compound of Formula 32 can be carried out under similar conditions as that of conversion of compound of Formula 6 to give a compound of Formula 7.

The reaction of a compound of Formula 32 with a compound of Formula R¹¹(CH₂)₄NH₂ to form a compound of Formula 33 can be carried out similarly as conversion of a compound of Formula 7 to give a compound of Formula 8.

The deprotection of a compound of Formula 33 to form a compound of Formula 34 can be carried out similarly as deprotection of a compound of Formula 8 to give a compound of Formula 9.

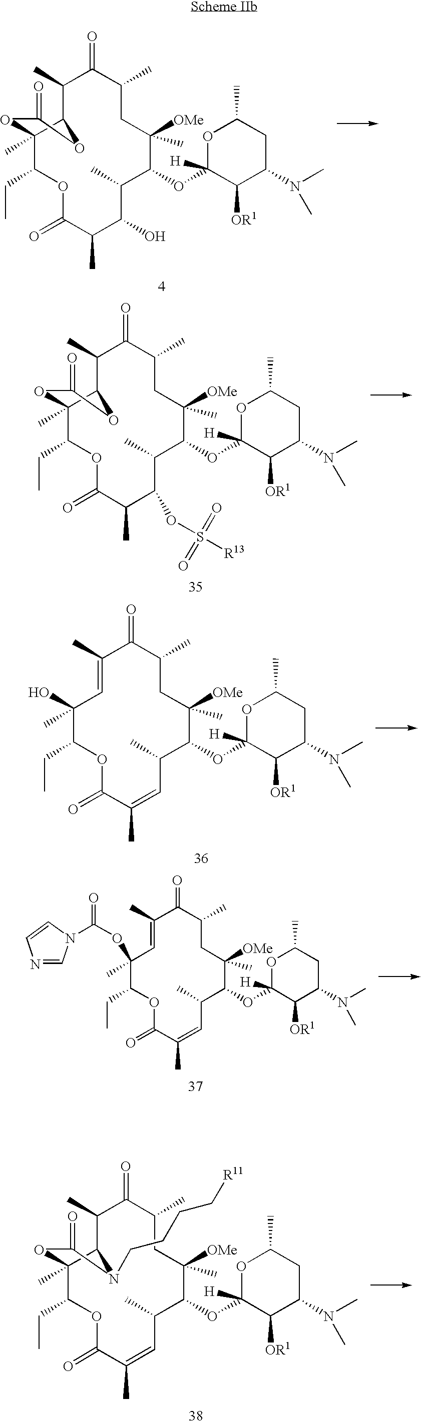

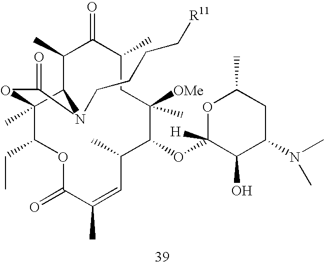

Compounds of Formula 39 can be prepared according to Scheme IIb. Thus, compound of Formula 4 is reacted with a compound of Formula R¹³SO₂Cl (R¹³ is aryl or alkyl) to give a compound of Formula 35 which is then converted to a compound of Formula 36. The compound of Formula 36 is reacted with N,N′-carbonyldiimidazole to form a compound of Formula 37. The compound of Formula 37 is further reacted with a compound of Formula R¹¹(CH₂)₄NH₂ to give a compound of Formula 38 which is then deprotected to give a compound of Formula 39. The compound of Formula 39 can further be converted into its salt by following the conventional method well known in the prior art.

The reaction of a compound of Formula 4 with a compound of Formula R¹³SO₂Cl to give a compound of Formula 35 can be carried out in one or more organic bases, for example, pyridine, triethylamine, trimethylamine, tributylamine, N-ethyldiisopropylamine, 4-N,N-dimethylaminopyridine, N-methylmorpholine or 2,6-lutidine in polar aprotic solvents (dimethylsulfoxide, dimethylformamide, acetone, tetrahydrofuran, acetonitrile) or non-polar solvents (dichloromethane, toluene, dichloroethane, ether).

Conversion of a compound of Formula 35 to give a compound of Formula 36 can be carried out under similar condition as that of conversion of compound of Formula 4 to give a compound of Formula 5.

The reaction of compound of Formula 36 with N,N′-carbonyldiimidazole to form a compound of Formula 37 can be carried out under similar conditions as that of conversion of compound of Formula 6 to give a compound of Formula 7.

The reaction of a compound of Formula 37 with a compound of Formula R¹¹(CH₂)₄NH₂ to form a compound of Formula 38 can be carried out similarly as conversion of a compound of Formula 7 to give a compound of Formula 8.

The deprotection of a compound of Formula 38 to form a compound of Formula 39 can be carried out similarly as deprotection of a compound of Formula 8 to give a compound of formula 9.

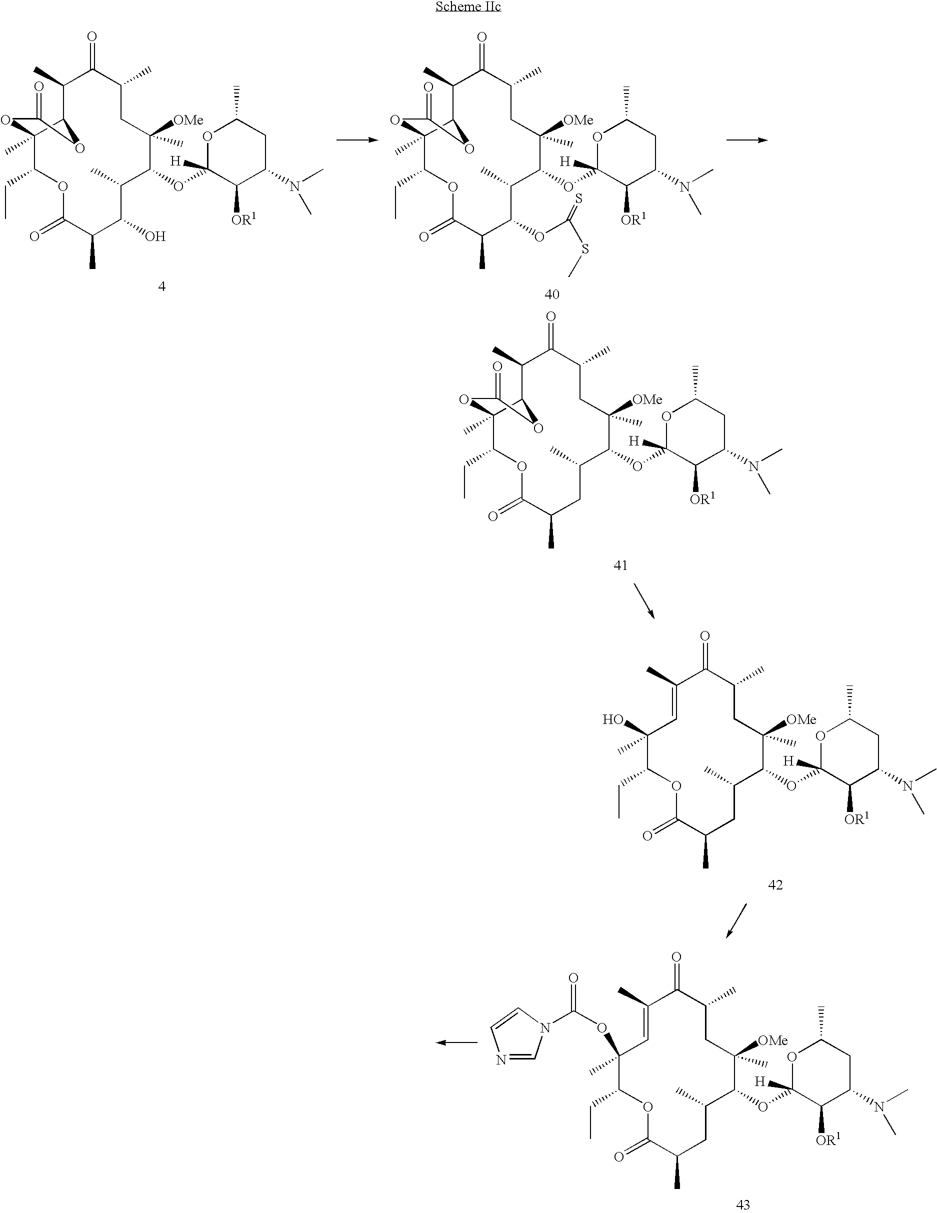

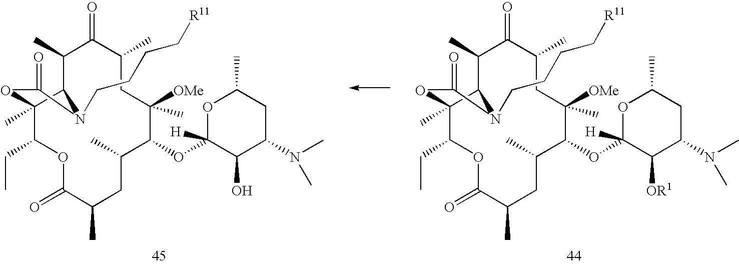

Compound of Formula 45 can be prepared according to Scheme IIc. Thus, compound of Formula 4 is converted to corresponding S-methyl dithiocarbonate derivative of Formula 40 which is then reduced to a compound of Formula 41. The compound of Formula 41 in presence of organic base forms a compound of Formula 42. The compound of Formula 42 is reacted with N,N′-carbonyldiimidazole to form a compound of Formula 43. The compound of Formula 43 is further reacted with a compound of Formula R¹¹(CH₂)₄NH₂ to give a compound of Formula 44 which is then deprotected to give a compound of Formula 45. The compound of Formula 45 can further be converted into its salt by following the conventional method well known in the prior art.

Derivatization of compound of Formula 4 to form S-methyl dithiocarbonate derivative of Formula 40 can be carried out in polar aprotic solvent, for example, tetrahydrofuran, acetonitrile, 1,4-dioxane or dimethylformamide, in the presence of a base for example sodium hydride or potassium hydride with carbon disulphide and methyl iodide.

The reduction of a compound of Formula 40 to give a compound of Formula 41 can be carried out in non-polar solvents for example toluene or benzene with reducing agent for example, tributyl tin hydride and azoisobutyronitrile.

The reaction of a compound of Formula 41 with an organic base, for example, tetramethyl guanidine, pyridine or trimethylamine to form a compound of Formula 42 can be carried out similarly as that of conversion of compound of Formula 4 to form a compound of Formula 5.

The reaction of compound of Formula 42 with N,N′-carbonyldiimidazole to form a compound of Formula 43 can be carried out under similar conditions as that of conversion of compound of Formula 6 to give a compound of Formula 7.

The reaction of a compound of Formula 43 with a compound of Formula R¹¹(CH₂)₄NH₂ to form a compound of Formula 44 can be carried out similarly as conversion of a compound of Formula 7 to give a compound of Formula 8.

The deprotection of a compound of Formula 44 to form a compound of Formula 45 can be carried out similarly as deprotection of a compound of Formula 8 to give a compound of formula 9.

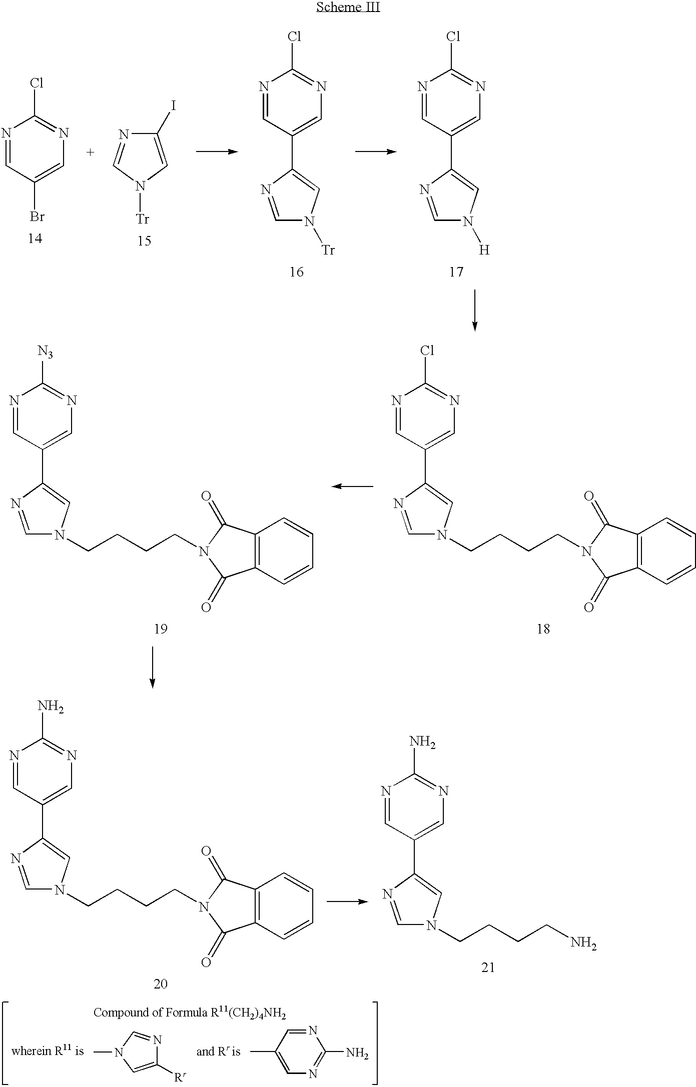

Compound of Formula 21 can be prepared according to scheme III. Thus, the compound of Formula 14 is coupled with a compound of Formula 15 to form a compound of Formula 16 (wherein Tr is trityl). The compound of Formula 16 is deprotected to form a compound of Formula 17. The compound of Formula 17 is reacted with N-(4-bromobutyl)-phthalimide to form a compound of Formula 18. The compound of Formula 18 is reacted with alkali metal azide to form a compound of Formula 19. The compound of Formula 19 is reduced to form a compound of Formula 20. The compound of Formula 20 is reacted with hydrazine monohydrate to form a compound of Formula 21.

The reaction of a compound of Formula 14 with organo zinc compound (prepared in situ by transmetallation of organo magnesium compound of Formula 15 with zinc chloride) to form a compound of Formula 16 can be carried out in the presence of palladium catalysts, for example, palladium (II) acetate, palladium (II) trifluoroacetate, palladium (II) propionate, tetrakis(triphenylphosphine) palladium (0), bis(dibezylidineacetone) palladium (0), bis(triphenylphosphine) palladium (II) chloride or mixture thereof, in one or more ether solvents, for example, diethyl ether, dimethyl ether, tetrahydrofuran, dioxane or mixture thereof.

The deprotection of a compound of Formula 16 to form a compound of Formula 17 can be carried out in the presence of one or more mineral acids, hydrochloric acid, hydrobromic acid, hydroiodic acid or mixture thereof, in one or more protic polar solvents, for example, methanol, ethanol, propanol, isopropanol, butanol, water or mixture thereof.

The reaction of a compound of Formula 17 with N-(4-bromobutyl)-phthalimide to form a compound of Formula 18 can be carried out in the presence of sodium hydride in one or more solvents, for example, aprotic polar solvents (e.g., dimethylformamide or dimethylsulfoxide), ketones (e.g., acetone or ethyl methyl ketone), nitriles (e.g., acetonitrile or propionitrile) or mixture thereof.

The reaction of a compound of Formula 18 with alkali metal azides for example, sodium azide or lithium azide to form a compound of Formula 19 can be carried out in-one or more solvents, for example, aprotic polar solvents (e.g., dimethylformamide or dimethylsulfoxide), ketones (e.g., acetone or ethyl methyl ketone), nitriles (e.g., acetonitrile or propionitrile) or mixture thereof.

The reduction of a compound of Formula 19 form a compound of Formula 20 can be carried out in the presence of one or more reducing agents, for example, raney nickel/hydrogen, palladium-carbon/hydrogen, platinum/hydrogen and ammonia or mixture thereof, in one or more solvents, for example, chlorinated solvents (e.g., chloroform, dichloromethane or dichloroethane), protic polar solvents (methanol, ethanol, propanol, isopropanol or water) or mixture thereof.

The reaction of a compound 20 with hydrazine monohydrate to form a compound of Formula 21 can be carried out in one or more protic polar solvents, for example, methanol, ethanol, propanol, isopropanol, butanol, water or mixture thereof

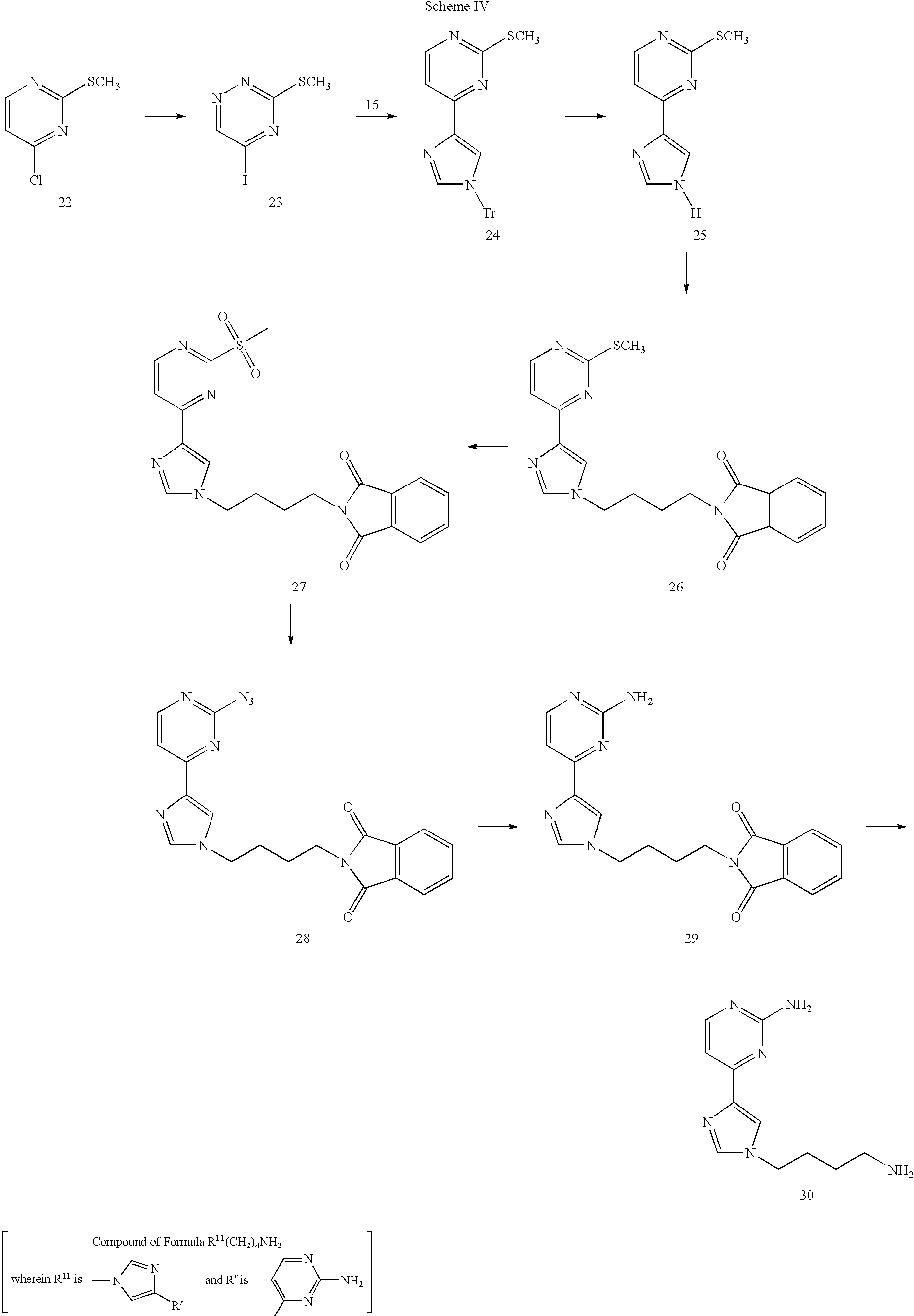

Compound of Formula 30 can be prepared according to scheme IV. Thus, the compound of Formula 22 is reacted with hydroiodic acid to form a compound of Formula 23. The compound of Formula 23 is coupled with a compound of Formula 15 to form a compound of Formula 24(wherein Tr is trityl). The compound of Formula 24 is deprotected to form a compound of Formula 25 The compound of Formula 25 is reacted with N-(4-bromobutyl)-phthalimide to form a compound of Formula 26. The compound of Formula 26 is oxidized to form a compound of Formula 27. The compound of Formula 27 is reacted with sodium azide to form a compound of Formula 28. The compound of Formula 28 is reduced to form a compound of Formula 29. The compound of Formula 29 is reacted with hydrazine monohydrate to form a compound of Formula 30.

The reaction of a compound of Formula 22 with hydroiodic acid to form a compound of Formula 23 can be carried out at a temperature ranging from 25 to 50° C.

The reaction of a compound of Formula 23 with a compound of Formula 15 to form a compound of Formula 24 can be carried out under similar conditions as that of reaction of a compound of Formula 14 with a compound of Formula 15 to form a compound of Formula 16.

The deprotection of a compound of Formula 24 form a compound of Formula 25 can be carried out similarly as deprotection of a compound of Formula 16 to give a compound of Formula 17.

The reaction of a compound of Formula 25 with N-(4-bromobutyl)-phthalimide to form a compound of Formula 26 can be carried out in the presence of one or more bases, for example, alkali metal bases (e.g., sodium hydrogen carbonate, sodium acetate, sodium thiosulphate, potassium carbonate, cesium carbonate or sodium hydride), alkaline earth metal bases (e.g., calcium carbonate, or calcium hydroxide) or mixture thereof. In one or more solvents, for example, aprotic polar solvents (e.g., dimethylformamide or dimethylsulfoxide), ketones (e.g., acetone or ethyl methyl ketone), nitriles (e.g., acetonitrile or propionitrile) or mixture thereof.

The oxidation of a compound of Formula 26 to form a compound of Formula 27 can be carried out in the presence of one or more peroxyacids, for example, peracetic acid, magnesium monoperoxyphthalate, potassium peroxomonosulfate, meta-chloroperbenzoic acid or mixture thereof, in one or more aprotic polar solvents (e.g., dimethylsulfoxide or dimethylformamide), chlorinated solvents (e.g., dichloromethane, dichloroethane, chloroform or carbon tetrachloride).

The reaction of a compound of Formula 27 with alkali metal azides for example, sodium azide or lithium azide to form a compound of Formula 28 can be carried out under similar conditions as that of conversion of compound of Formula 18 to form a compound of Formula 19.

The reduction of a compound of Formula 28 to form a compound of Formula 29 can be carried out similarly as that of reduction of a compound of Formula 19 to form a compound of Formula 20.

The reaction of a compound 29 with hydrazine monohydrate to form a compound of Formula 30 can be carried out under similar conditions as that of conversion of a compound of Formula 20 to give a compound of Formula 21.

In the above schemes, where specific reagents, for example, bases, acids, oxidizing agents, reducing agents, solvents, etc., are described, it is to be understood that other reagents, e.g., bases, acids, oxidizing agents, reducing agents, solvents, etc., known to one of ordinary skill in the art may be used. Similarly, reaction temperatures and durations may be adjusted according to the desired needs without undue experimentation and well within the abilities of one of ordinary skill in the art. All the epimers, unless otherwise specified in the above schemes are also encompassed within the scope of the invention.

Compounds of the present invention useful for such purpose are listed below:
R^{r} is Formula “X”; L is (CH₂)₄; R is Formula A [P and Q together form oxo group, R¹=H, R²=Et, Z=O, R′=Me, T=H], (Compound No. 1),
R^{r} is Formula “Y”; L is (CH₂)₄; R is Formula A [P and Q together form oxo group, R¹=H, R²=Et, Z=O, R′=Me, T=H], (Compound No. 2),
R^{r} is Formula “X”; L is (CH₂)₄; R is Formula A [P═H, Q=O-(2-pyridyl acetyl), R¹=H, R²=Et, Z=O, R′=Me, T=H], (Compound No. 3),
R^{r} is Formula “X”; L is (CH₂)₄; R is Formula A [P═H, Q=O-(3-pyridyl acetyl), R¹=H, R²=Et, Z=O, R′=Me, T=H], (Compound No. 4),
R^{r} is Formula “X”; L is (CH₂)₄; R is Formula A [P═H, Q=O-(2-fluorophenyl acetyl), R¹=H, R²=Et, Z=O, R′=Me, T=H], (Compound No. 5),
R^{r} is Formula “X”; L is (CH₂)₄; R is Formula A [P═H, Q=O-(3-fluorophenyl acetyl), R¹=H, R²=Et, Z=O, R′=Me, T=H], (Compound No. 6),
R^{r} is Formula “Y”; L is (CH₂)₄; R is Formula A [P═H, Q=O-(3-fluorophenyl acetyl), R¹=H, R²=Et, Z=O, R′=Me, T=H], (Compound No. 7),
R^{r} is Formula “Y”; L is (CH₂)₄; R is Formula A [P═H, Q=O-(2-fluorophenyl acetyl), R¹=H, R²=Et, Z=O, R′=Me, T=H], (Compound No. 8),
R^{r} is Formula “Y”; L is (CH₂)₄; R is Formula A [P═H, Q=O-(3-pyridyl acetyl), R¹=H, R²=Et, Z=O, R′=Me, T=H], (Compound No. 9),
R^{r} is Formula “Y”; L is (CH₂)₄; R is Formula A [P═H, Q=O-(2-pyridyl acetyl), R¹=H, R²=Et, Z=O, R′=Me, T=H], (Compound No. 10),
R^{r} is Formula “X”; L is (CH₂)₄; R is Formula A [P and Q together form oxo group, R¹=H, R²=Et, Z=O, R′=Me, T=F], (Compound No. 11),
R^{r} is Formula “X”; L is (CH₂)₄; R is Formula A [P═H, Q=H, R¹=H, R²=Et, Z=O, R′=Me, T=H], (Compound No. 12),
R^{r} is Formula “X”; L is (CH₂)₄; R is Formula D [R¹=H, R²=Et, Z=O, R′=Me] (Compound No. 13), or
pharmaceutically acceptable salts, pharmaceutically acceptable solvates, enantiomers, diastereomers or polymorphs thereof.

The compounds disclosed herein are pharmacologically active against gram-positive, gram-negative and anaerobic bacteria and accordingly, are useful as antibacterial agents for treating bacterial infections in a patient in need thereof, for example, in a human or an animal. Because of their antibacterial activity, the compounds described herein may be administered to an animal for treatment orally, topically, rectally, internasally, or by parenteral route. Pharmaceutical compositions disclosed herein comprise pharmaceutically effective amounts of compounds described herein formulated together with one or more pharmaceutically acceptable carriers, excipients or diluents.

Solid form preparations for oral administration include capsules, tablet, pills, powder, granules, cachets and suppositories. For solid form preparations, active compounds can be mixed with one or more inert, pharmaceutically acceptable excipients or carrier, for example, sodium citrate, dicalcium phosphate and/or fillers or extenders (for example, starches, lactose, sucrose, glucose, mannitol, silicic acid or mixtures thereof); binders, for example, carboxymethylcellulose, alginates, gelatins, polyvinylpyrrolidinone, sucrose, acacia or mixtures thereof, disintegrating agents, for example, agar-agar, calcium carbonate, potato starch, alginic acid, certain silicates, sodium carbonate or mixtures thereof, absorption acceletors, for example, quaternary ammonium compounds; wetting agents, for example, cetyl alcohol, glycerol mono stearate or mixtures thereof, adsorbants, for example, Kaolin; lubricants, for example, talc, calcium stearate, magnesium stearate, solid polyethyleneglycol, sodium lauaryl sulfate or mixtures thereof.

Capsules, tablets or pills may also comprise buffering agents.

Tablets, capsules, pills or granules can be prepared using one or more coatings or shells, for example, enteric coatings or other coatings known to one of ordinary skill in the art.

Liquid form preparations for oral administration include pharmaceutically acceptable emulsions, solutions, suspensions, syrups or elixirs. In such liquid form preparations, active compounds can be mixed with water or one or more other solvents, solubilizing agents or emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil, glycerol, fatty acid esters of sorbitan or mixtures thereof. Oral compositions can also include one or more adjuvants, for example, wetting agents, emulsifying agents, suspending agents, sweetening agents, flavouring agents, perfuming agents or mixtures thereof.

Injectable preparations, for example, sterile injections, aqueous suspensions may be formulated according to methods known to one of ordinary skill in the art, and in particular, using one or more suitable dispersing or wetting and suspending agents. Acceptable vehicles and solvents that may be employed include one or more of water, Ringer's solution, isotonic sodium chloride or mixtures thereof.

Dosage forms for tropical or transdermal administration of a compound of the present invention include ointments, pastes, creams, lotions, gels, powders, solutions, sprays, inhalants or patches. Active compounds can be admixed under sterile condition with one or more pharmaceutically acceptable carriers and optionally any preservatives or buffers as may be required. Ophthalmic formulations, eardrops, eye ointments, powders and solutions are also encompassed within the scope of this invention.

Pharmaceutical preparations may be in unit dosage form. In unit dosage form, the preparations can be subdivided into unit doses containing appropriate quantities of active components. Unit dosage forms can be packaged preparations containing discrete capsules, powders, in vials or ampoules, ointments, capsules, sachets, tablets, gels, creams or any combination and number of such packaged forms.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example 1

### Preparation of 5-[1-(4-Amino-butyl)-1H-imidazol-4-yl]-pyrimidin-2-ylamine

### Step-1: Preparation of 2-chloro-5-(1-trityl-1H-imidazol-4-yl)-pyrimidine

Ethyl magnesium bromide (1M) in tetrahydrofuran (12.66 mL, 12.66 mmol) was added to the solution of 4-iodo-1-trityl-1H-imidazole (4.6 g, 10.55 mmol) in freshly distilled dry tetrahydrofuran (46.0 mL) at an ambient temperature and the reaction mixture was stirred for about 90 minutes. Zinc chloride (1M) (12.66 mL, 12.66 mmol) was added at an ambient temperature and was stirred for another about 90 minutes. The reaction mixture was degassed for about 20 minutes. Tetrakis(triphenylphosphine) palladium (0.61 g, 0.527 mmol) and 5-bromo-2-chloro pyrimidine (2.24 g, 11.6 mmol) was added and the reaction mixture was stirred for about 12-14 hours at about 70° C. The reaction mixture was cooled and diluted with dichloromethane, washed with aqueous solution of EDTA (PH=9), brine, dried over anhydrous sodium sulphate, filtered and concentrated to form the solid, which was purified by column chromatography using ethyl acetate-hexane as eluents. Yield: 3.0 g

### Step-2: Preparation of 2-chloro-35-(1H-imidazol-4-yl)-pyrimidine

Hydrochloric acid (Conc., 2.0 mL) was added to a solution of 2-chloro-5-(1-trityl-1H-imidazol-4-yl)-pyrimidine (6.2 g) in ethanol (62 mL) and heated at about 45° C. for about 3 hours. The reaction mixture was cooled to about 30° C. The solvent was evaporated under reduced pressure. The resulting residue was basified with saturated sodium bicarbonate solution (pH=7.5), and then extracted with ethyl acetate. Organic layer was washed with water, brine, dried over anhydrous sodium sulphate and concentrated to form the crude product, which was purified by column chromatography. Yield (2.7 g)

### Step-3: Preparation of 2-[4-[4-(2-chloro-pyrimidin-5-yl)-imidazol-1-yl]-butyl]-isoindole-1,3-dione

Sodium hydride (60%, 0.9 g, 22.37 mmol) was added to a stirred solution of 2-chloro-5-(1H-imidazol-4-yl)-pyrimidine (2.7 g 14.91 mmol) in N,N-dimethyl formamide (27.0 mL) in portions at 0° C. and the reaction mixture was stirred for about 30 minutes. To the reaction mixture was added N-(4-bromobutyl)-phthalimide (5.9 g, 20.88 mmol) and stirred for about 2 hours at about 30° C. The reaction mixture was poured into ice-cold water, and extracted with ethyl acetate. The organic layer was washed with water, brine, dried over anhydrous sodium sulphate and concentrated to form crude product (3.5 g), which was purified by column chromatography using ethyl acetate-hexane as eluents.

### Step-4: Preparation of 2-[4-[4-(2-azido-pyrimidin-5-yl)-imidazol-1-yl]-butyl]-isoindole-1,3-dione

Sodium azide (0.52 g, 7.87 mmol) was added to a stirred solution of 2-{4-[4-(2-Chloro-pyrimidin-5-yl)-imidazol-1-yl]-butyl}-isoindole-1,3-dione (1.5 g, 3.937 mmol) in dimethyl sulfoxide (15 mL) and was heated at about 90° C. for about 8.0 hours. The reaction mixture was cooled and poured into water, filtered and dried under reduced pressure to form the title product (1.1 g).

### Step-5: Preparation of 2-[4-[4-(2-Amino-pyrimidin-5-yl)-imidazol-1-yl]-butyl]-isoindole-1,3-dione

2-{4-[4-(2-Azido-pyrimidin-5-yl)-imidazol-1-yl]-butyl}-isoindole-1,3-dione (1.0 g, 27.8 mmol) and palladium on carbon (10%) were taken in dichloromethane and methanol (8:2, 10 mL). The reaction mixture was stirred at about 30° C. under hydrogen atmosphere for about 2 hours. The reaction mixture was filtered, concentrated under reduced pressure to form the title compound (0.9 g).

### Step-6: Preparation of 5-[1]-(4-Amino-butyl)-1H-imidazol-4-yl]-pyrimidin-2-ylamine

Hydrazine monohydrate (0.72 mL, 14.88 mmol) was added to a solution of 2-{4-[4-(2-Amino-pyrimidin-5-yl)-imidazol-1-yl]-butyl}-isoindole-1,3-dione (0.9 g, 2.48 mmol) in ethanol (10.0 mL) and the reaction mixture was heated to about 60° C. for about 3 hours. The reaction mixture was cooled to about 30° C. and digested with dichloromethane, filtered on celite bed, concentrated under reduced pressure to form the crude product. The crude product was purified by column chromatography using methanol-dichloromethane-triethylamine as eluant to form the title product (0.45 g).

Mass (M+1): 233.3

¹H NMR (CDCl₃+MeOH-d₄): δ1.4 (m, 2H), 1.78 (m, 2H), 2.63 (t, 2H), 3.21 (s, 2H), 3.98 (t, 2H), 5.1 (s, 2H), 7.4 (s, 1H), 7.64 (s, 1H), 8.49 (s, 2H).

### Example 2

### Preparation of 4-[1-(4-aminobutyl)-1H-imidazol-4-yl]-pyrimidin-2-yl amine

### Step-1: Preparation of 4-Iodo-(2-methylthio)pyrimidine

Hydroiodic acid (57%) was added to 4-chloro-(2-methylthio)pyrimidine at about 30° C. and stirred for about 72 hours in dark. The reaction mixture was filtered through sintered funnel, dissolved in water and basified with sodium bicarbonate and then extracted with chloroform. The chloroform layer was washed with aqueous sodium thiosulphate solution, water, brine, dried over anhydrous sodium sulphate, concentrated and crystallized from hexane to form the title product.

### Step-2: Preparation of 2-methylsulfanyl-4-(1-trityl-1H-imidazol-4-4yl)-pyrimidine

Ethyl magnesium bromide (1M) in tetrahydrofuran (82.6 mL, 82.6 mmol) was added to a solution of 4-iodo-1-trityl-1H-imidazole (30.0 g, 68.8 mmol) in dry tetrahydrofuran (300 mL) in three-necked RB flask at room temperature and was stirred for about 90 minutes. The solution of zinc chloride (1M, 82.6 mL, 82.6 mmol) was added to the reaction mixture at an ambient temperature and was stirred for about 90 minutes. The reaction mixture was degassed for about 30 minutes. Tetrakis-(triphenylphosphine)-palladium (0) (4.8 g, 4.1 mmol) and 4-iodo-(2-methylthio)-pyrimidine (20.8 g, 82.6 mmol) were added to reaction mixture and refluxed for about 14 hours. The reaction mixture was cooled, diluted with dichloromethane. Aqueous solution of EDTA was added to it and stirred for about 1 hour. The organic layer was separated, washed with water, brine, dried over anhydrous sodium sulphate, filtered, concentrated to form the crude product. The crude product was purified by column chromatography to form the title product (15.4 g).

### Step-3: Preparation of 4-(1H-imidazol-4-yl)-2-methylsulfanyl-pyrimidine

Hydrochloric acid (Conc., 5 mL) was added to a solution of 2-methylsulfanyl-4-(1-trityl-1H-imidazol-4-4-yl)-pyrimidine (15.2 g) in ethanol (100 mL) and heated at about 60° C. for about 3 hours. The reaction mixture was cooled to about 30° C. Ethanol was evaporated under reduced pressure. The residue was basified by aqueous saturated solution of sodium bicarbonate (pH=7.5), extracted with ethyl acetate. The organic layer was washed with water, brine, dried over anhydrous sodium sulphate, and concentrated to form the crude product. The crude product was purified by column chromatography to form the title compound (3.7 g).

### Step-4: Preparation of 2-[4-[4-(2-methylsulfanyl-pyrimidin-4-yl)-imidazol-1-yl]-butyl]-isoindole-1,3-dione

Sodium hydride (60%, 1.1 g, 27.3 mmol) was added to a stirred solution of 4-(1H-imidazol-4-yl)-2-methylsulfanyl-pyrimidine (3.5 g, 18.2 mmol) in N,N-dimethyl formamide (30 mL) in portions at about 0° C., and the reaction mixture was stirred for about 20 minutes. N-(4-bromobutyl)-phthalimide (6.2 g, 21.9 mmol) was added to it and stirred for about 3 hours at about 30° C. The reaction mixture was poured into ice-cold water, extracted with ethyl acetate, washed with water, brine, dried over anhydrous sodium sulphate, and concentrated to form the title product (4.5 g).

### Step 5: Preparation of 2-[4-[4-(2-methanesulfonyl-pyrimidin-4-yl)-imidazol-1-yl]-butyl]-isoindole-1,3-dione

m-Chloro perbenzoic acid (70-77%, 6.6 g, 26.7 mmol) was added to a cooled solution of 2-{4-[4-(2-methylsulfanyl-pyrimidin-4-yl)-imidazol-1-yl]-butyl}-isoindole-1,3-dione (3.5 g, 8.9 mmol) in dichloromethane (30 mL) in portions at about 0° C. The reaction mixture was stirred for 5 minutes and quenched by the addition of aqueous sodium bisulphate solution. The resulting mixture was stirred for 5 minutes then was basified by aqueous sodium bicarbonate solution. Organic layer was diluted with dichloromethane, separated, washed with water, brine, dried over anhydrous sodium sulphate and concentrated. The crude product was purified by column chromatography to give title compound (1.92 g).

### Step-6: Preparation of 2-[4-[4-(2-Azido-pyrimidin-4-yl)-imidazol-1-yl]-butyl]-isoindole-1,3-dione

Sodium azide (581 mg, 8.94 mmol) was added to a solution of 2-{4-[4-(2-methanesulfonyl-pyrimidin-4-yl)-imidazol-1-yl]-butyl}-isoindole-1,3-dione (1.9 g, 4.5 mmol) in dimethylsulfoxide (10 mL) and heated at about 80° C. for about 1 hour. The reaction mixture was cooled to about 30° C., poured into ice-cold water, extracted with dichloromethane, washed with water, brine, dried over anhydrous sodium sulphate, and concentrated to form the title product (1.72 g).

### Step-7: Preparation of 2-[4-[4-(2-Amino-pyrimidin-4-yl)-imidazol-1-yl]-butyl]-isoindole-1,3-dione

Palladium on carbon (10%, 200 mg) was added to a solution of 2-{4-[4-(2-Azido-pyrimidin-4-yl)-imidazol-1-yl]-butyl}-isoindole-1,3-dione (1.7 g, 4.4 mmol) in a mixture of dichloromethane (32 mL) and methanol (8 mL). The reaction mixture was stirred at about 30° C. under hydrogen atmosphere for about 16 hours. The reaction mixture was filtered, and concentrated under reduced pressure to form the title product (0.9 g).

### Step-8: Preparation of 4-[1-(4-Amino-butyl)-1H-imidazol-4-yl]-pyrimidin-2-ylamine

Hydrazine monohydrate (0.4 mL, 8 mmol) was added to a solution of 2-{4-[4-(2-amino-pyrimidin-4-yl)-imidazol-1-yl]-butyl}-isoindole-1,3-dione (970 mg, 2.7 mmol) in ethanol (15 mL) and the reaction mixture was heated at about 60° C. for about 3 hours. The reaction mixture was cooled at about 30° C. and digested with dichloromethane, filtered on celite bed, concentrated under reduced pressure to form the crude product. The crude product was purified by column chromatography using methanol-dichloromethane-triethylamine as eluant to form the title product (503 mg).

Mass (M+1): 233.2

¹H NMR (CDCl₃+DMSO-d₆): δ1.4 (m, 2H), 1.86 (m, 2H), 2.57 (s, 2H), 2.69 (t, 2H), 4.04 (t, 2H), 5.09 (s, 2H), 7.08 (d, 1H), 7.61 (s, 1H), 7.71 (s, 1H), 8.21 (d, 1H).

### Example 3

### Preparation of a Compound of Formula 2

Clarithromycin (25 g, 33.4 mmol) was added to an aqueous solution of hydrochloric acid at an ambient temperature in portion wise. The reaction mixture was neutralized with solid sodium bicarbonate and the aqueous layer was extracted with ethyl acetate. The organic layer was washed with water, brine, dried over anhydrous sodium sulphate, and the solvent was removed under reduced pressure to form the crude product. The crude product was crystallized from ethyl acetate and hexane to form the title compound.

### Example 4

### Preparation of Compound of Formula 3

Benzoic anhydride (2.5 equiv.) followed by triethylamine (6 equiv.) was added to a solution of compound of Formula 2 (1 equiv.) in dichloromethane and stirred at an ambient temperature for about 40 hours. The reaction was quenched by sodium bicarbonate solution. The aqueous layer was extracted with dichloromethane, washed successively with water, brine, dried over anhydrous sodium sulphate, and the solvent was removed under reduced pressure to form the crude product. The crude product was crystallized from a mixture of ethyl acetate and hexane to yield the title compound.

### Example 5

### Preparation of Compound of Formula 4

Triphosgene (1.5 equiv.) was added to a solution of compound of Formula 3 (1 equiv.) in dichloromethane. Pyridine (15 equiv.) was added to it slowly. The reaction mixture was stirred for about 4 hours followed by quenching with ice-cold water. The reaction mixture was diluted with dichloromethane, washed with water, brine, dried over anhydrous sodium sulphate, and concentrated under reduced pressure to yield the title product.

### Example 6

### Preparation of Compound of Formula 5

Tetramethyl guanidine (2.2 equiv.) was added to a solution of compound of Formula 4 (1 equiv.) in dimethylformamide and the reaction mixture was heated at about 70° C. followed by stirring for about 10 hours. The reaction mixture was cooled to an ambient temperature, extracted with ethyl acetate, washed with water, brine, dried over anhydrous sodium sulphate, and concentrated under reduced pressure to yield the title product.

### Example 7

### Preparation of Compound of Formula 6

Dess-Martine Periodinane (2.5 equiv.) was added to a solution of compound of Formula 5 (1 equiv.) in dichloromethane and refluxed for about an hour. The reaction mixture was cooled to an ambient temperature and quenched by aqueous saturated solution of potassium carbonate followed by saturated sodium thiosulphate solution and stirred. Aqueous layer was separated and extracted with dichloromethane, washed with water, brine, dried over anhydrous sodium sulphate and concentrated under reduced pressure to yield the title product.

### Example 8

### Preparation of Compound of Formula 7

N,N′-carbonyldiimidazole (3 equiv.) was added to a solution of compound of Formula 6 (1 equiv.) in a mixture of dimethylformamide and tetrahydrofuran (3:2) at an ambient temperature. The reaction mixture was cooled. Sodium hydride (3 equiv.) was added to it in portion wise and stirred. The reaction mixture was quenched by water, extracted with ethyl acetate, washed with water, brine, dried over anhydrous sodium sulphate, and concentrated under reduced pressure to yield the title product.

### Example 9

### Preparation of Compound of Formula 8

Compound of Formula 7 (1 equiv.) and R¹¹(CH₂)₄NH₂ (2 equiv.) were taken in water in acetonitrile and heated at about 70° C., stirred for about 20 hours. The reaction mixture was cooled to an ambient temperature and acetonitrile was removed under reduced pressure. The residue was taken in ethyl acetate, washed with water, brine, dried over anhydrous sodium sulphate, filtered, and concentrated under reduced pressure to form the crude product. The crude product was purified by silica gel column chromatography (thoroughly neutralized with triethylamine) using 25-30% acetone in hexane to form yield the title product.

### Example 10

### Preparation of Compound of Formula 9

A solution of compound of Formula 8 in methanol was refluxed for about 20 hours. The reaction mixture was cooled to an ambient temperature and methanol was evaporated under reduced pressure to form the crude product., which was purified by silica gel column chromatography using 2-10% methanol in dichloromethane as eluants to yield the title compound.

The following compounds were prepared following the procedure described in Example 3 to 10
Compound No. 1: 11,12-dideoxy-3-O-decladinosyl-6-O-methyl-3-oxo-12,11-[oxycarbonyl-((4-(4-(2-amino-pyrimidin)-5-yl)-imidazol-1-yl)-butyl)-imino)]erythromycin A; Mass (m/z) (M+1): 828.7, m.pt. 131-132,
Compound No. 2: 11,12-dideoxy-3-O-decladinosyl-6-O-methyl-3-oxo-12,11-[oxycarbonyl-((4-(4-(2-amino-pyrimidin)-4-yl)-imidazol-1-yl)-butyl)-imino)]erythromycin A (Compound No. 2); Mass (m/z) (M+1): 828.7, m.pt. 132-133.

### Example 11

### Preparation of Compound of Formula 10

Compound of Formula R³CH₂COOH (2.5 equiv.), 4-N-dimethylaminopyridine (2.5 equiv) and N,N′-dicyclohexylcarbodiimide (2.5 equiv) were added to a solution of compound of Formula 5 (1 equiv) in dichloromethane. Pyridine (4 equiv.) was added to it, stirred and filtered through celite bed. The filtrate was washed with water, brine, dried over anhydrous sodium sulphate and concentrated under reduced pressure to form the crude product. The crude product was purified by silica gel column chromatography (thoroughly neutralized with triethylamine) using 10-20% acetone in hexane to afford the title compound.

### Example 12

### Preparation of Compound of Formula 11

N,N′-carbonyldiimidazole (3 equiv.) was added to a solution of compound of Formula 10 (1 equiv.) in a mixture of dimethylformamide and tetrahydrofuran (3:2) at an ambient temperature. The reaction mixture was cooled. Sodium hydride (3 equiv.) was added to it in portion wise and stirred. The reaction mixture was quenched by water, extracted with ethyl acetate, washed with water, brine, dried over anhydrous sodium sulphate, and concentrated under reduced pressure to form the title product.

### Example 13

### Preparation of Compound of Formula 12

Compound of Formula 11 (1 equiv.) and R¹¹(CH₂)₄NH₂ (2 equiv.) were taken in water in acetonitrile and heated at about 70° C., stirred for about 20 hours. The reaction mixture was cooled to an ambient temperature and acetonitrile was removed under reduced pressure. The residue was taken in ethyl acetate and washed with water, brine, dried over anhydrous sodium sulphate, and filtered. The filtrate was concentrated under reduced pressure to form the crude product. The crude product was purified by silica gel column chromatography (thoroughly neutralized with triethylamine) using 25-30% acetone in hexane to afford the title compound.

### Example 14

### Preparation of Compound of Formula 13

Compound of Formula 12 was taken in methanol and refluxed for about 20 hours. The reaction mixture was cooled to an ambient temperature and methanol was evaporated under reduced pressure to form the crude product. The crude product was purified by silica gel column chromatography using 2-10% methanol in dichloromethane to afford the title compound.

The following compounds can be prepared following the procedure described in Example 11 to 14:
Compound No. 3: 11,12-Dideoxy-3-O-decladinosyl-3-O-(2-pyridyl acetyl)-6-O-methyl-12,11-[oxycarbonyl-((4-(4-(2-amino-pyrimidin)-5-yl)-imidazol-1-yl)-butyl)-imino)]erythromycin A,
Compound No. 4: 11,12-Dideoxy-3-O-decladinosyl-3-O-(3-pyridyl acetyl)-6-O-methyl-12,11-[oxycarbonyl-((4-(4-(2-amino-pyrimidin)-5-yl)-imidazol-1-yl)-butyl)-imino)]erythromycin A,
Compound No. 5: 11,12-Dideoxy-3-O-decladinosyl-3-O-(2-fluorophenyl acetyl)-6-O-methyl-12,11-[oxycarbonyl-((4-(4-(2-amino-pyrimidin)-5-yl)-imidazol-1-yl)-butyl)-imino)]erythromycin A,
Compound No. 6: 11,12-Dideoxy-3-O-decladinosyl-3-O-(3-fluorophenyl acetyl)-6-O-methyl-12,11-[oxycarbonyl-((4-(4-(2-amino-pyrimidin)-5-yl)-imidazol-1-yl)-butyl)-imino)]erythromycin A,
Compound No. 7: 11,12-Dideoxy-3-O-decladinosyl-3-O-(3-fluorophenyl acetyl)-6-O-methyl-12,11-[oxycarbonyl-((4-(4-(2-amino-pyrimidin)-4-yl)-imidazol-1-yl)-butyl)-imino)]erythromycin A,
Compound No. 8: 11,12-Dideoxy-3-O-decladinosyl-3-O-(2-fluorophenyl acetyl)-6-O-methyl-12,11-[oxycarbonyl-((4-(4-(2-amino-pyrimidin)-4-yl)-imidazol-1-yl)-butyl)-imino)]erythromycin A,
Compound No. 9: 11,12-Dideoxy-3-O-decladinosyl-3-O-(3-pyridyl acetyl)-6-O-methyl-12,11-[oxycarbonyl-((4-(4-(2-amino-pyrimidin)-4-yl)-imidazol-1-yl)-butyl)-imino)]erythromycin A,
Compound No. 10: 11,12-Dideoxy-3-O-decladinosyl-3-O-(2-pyridyl acetyl)-6-O-methyl-12,11-[oxycarbonyl-((4-(4-(2-amino-pyrimidin)-4-yl)-imidazol-1-yl)-butyl)-imino)]erythromycin A.

### Example 15

### Preparation of Compound of Formula 31

Potassium tert-butoxide (1.5 equiv.) was added to a solution of compound of Formula 6 (1 equiv.) in tetrahydrofuran at −15° C. The reaction mixture was stirred for 20 minutes and N-fluorobenzene sulfonamide (1.2 equiv.) in tetrahydrofuran was then added to the above mixture. The reaction mixture was stirred at −15° C. for about 3 hours, quenched by adding water, and extracted with ethyl acetate. The organic layer was washed with water followed by brine, dried over anhydrous sodium sulfate and concentrated under reduced pressure and purified by column chromatography to yield the title compound.

### Example 16

### Preparation of Compound of Formula 32

N,N′-carbonyldiimidazole (3 equiv.) was added to a solution of compound of Formula 31 (1 equiv.) in a mixture of dimethylformamide and tetrahydrofuran (3:2) at an ambient temperature. The reaction mixture was cooled. Sodium hydride (3 equiv.) was added to it in portion wise and stirred. The reaction mixture was quenched by water, extracted with ethyl acetate, washed with water, brine, dried over anhydrous sodium sulphate, and concentrated under reduced pressure to afford the title compound.

### Example 17

### Preparation of Compound of Formula 33

Compound of Formula 32 (1 equiv.) and R¹¹(CH₂)₄NH₂ (2 equiv.) were taken in water in acetonitrile and heated at about 70° C., stirred for about 20 hours. The reaction mixture was cooled to an ambient temperature and acetonitrile was removed under reduced pressure. The residue was taken in ethyl acetate and washed with water, brine, dried over anhydrous sodium sulphate, and filtered. The filtrate was concentrated under reduced pressure to form the crude product. The crude product was purified by silica gel column chromatography (thoroughly neutralized with triethylamine) using 25-30% acetone in hexane to afford the title compound.

### Example 18

### Preparation of Compound of Formula 34

Compound of Formula 33 was taken in methanol and refluxed for about 20 hours. The reaction mixture was cooled to an ambient temperature and methanol was evaporated under reduced pressure to form the crude product. The crude product was purified by silica gel column chromatography using 2-10% methanol in dichloromethane to afford the title compound.

The following compound was prepared following the procedure described in Examples 15 to 18:
Compound No. 11: 11,12-dideoxy-3-O-decladinosyl-2-Fluoro-6-O-methyl-3-oxo-12,11-[oxycarbonyl-(4-(4-(2-amino-pyrimidin-5-yl)-imidazol-1-yl)-butylimino)]erythromycin A

Mass (m/z) (M+1): 846.15

### Example 19

### Preparation of Compound of Formula 35

Triethylamine (1.2 equiv.) was added to a solution of compound of Formula 4 (1 equiv.) in dichloromethane at about 0° C. with stirring. Methane sulfonyl chloride (equiv) was added slowly to the above reaction mixture. After complete addition, reaction mixture was stirred for about 40-60 minutes at 0° C. The reaction mixture was quenched by drop wise addition of cold water at 0° C. Reaction mixture was diluted with dichloromethane and washed with water followed by brine, dried over anhydrous sodium sulphate and concentrated under reduced pressure to afford the title compound.

### Example 20

### Preparation of Compound of Formula 36

Tetramethyl guanidine (2.2 equiv.) was added to a solution of compound of Formula 35 (1 equiv.) in dimethylformamide and the reaction mixture was heated at about 80-90° C. with stirring for about 8 hours. The reaction mixture was cooled to an ambient temperature, water was added to the reaction mixture and extracted with ethyl acetate. The organic layer was washed with water, brine, dried over anhydrous sodium sulphate, and concentrated under reduced pressure to yield the title compound.

### Example 21

### Preparation of Compound of Formula 37

N,N′-carbonyldiimidazole (3 equiv.) was added to a solution of compound of Formula 36 (1 equiv.) in a mixture of dimethylformamide and tetrahydrofuran (3:2) at an ambient temperature. The reaction mixture was cooled to 0° C. Sodium hydride (3 equiv.) was added to it in portion wise and reaction mixture was stirred for about 30 minutes. The reaction mixture was quenched by water, extracted with ethyl acetate, washed with water, brine, dried over anhydrous sodium sulphate, and concentrated under reduced pressure to afford the title compound.

### Example 22

### Preparation of Compound of Formula 38

Compound of Formula 37 (1 equiv.) and R¹¹(CH₂)₄NH₂ (2 equiv.) were taken in 10% water in acetonitrile and heated at about 70° C., stirred for about 14 hours. The reaction mixture was cooled to an ambient temperature and acetonitrile was removed under reduced pressure. The residue was taken in ethyl acetate and washed with water, brine, dried over anhydrous sodium sulphate, and filtered. The filtrate was concentrated under reduced pressure to form the crude product. The crude product was purified by silica gel column chromatography (thoroughly neutralized with triethylamine) using 25-30% acetone in hexane to afford the title compound.

### Example 23

### Preparation of Compound of Formula 39

Compound of Formula 39 was taken in methanol and refluxed for about 12 hours. The reaction mixture was cooled to an ambient temperature and methanol was evaporated under reduced pressure to form the crude product. The crude product was purified by silica gel column chromatography using 2-10% methanol in dichloromethane to afford the title compound.

The following compound was prepared following the procedure described in Example 19 to 23:
Compound No. 12: 11,12-dideoxy-3-O-decladinosyl-3-deoxo-2,3-anhydro-6-O-methyl-3-oxo-12,11-[oxycarbonyl-(4-(4-(2-amino-pyrimidin-5-yl)-imidazol-1-yl)-butylimino)]erythromycin A

Mass (m/z) (M+1): 813, m.pt. 119° C.

### Example 24

### Preparation of Compound of Formula 40

Sodium hydride (1.1 equiv.) was added to a solution of compound of Formula 4 (1 equiv.) in tetrahydrofuran at about 0° C. to −5° C. with stirring. Carbon disulfide (1 equiv.) was added slowly, to the above reaction mixture. After 5 minutes methyl iodide (1 equiv.) was added and reaction mixture was stirred for about 30 minutes at 0° C. The reaction was quenched by drop wise addition of ice-cold water. Reaction mixture was diluted with ethylacetate and washed with water followed by brine, dried over anhydrous sodium sulphate and concentrated under reduced pressure to afford the title compound.

### Example 25

### Preparation of Compound of Formula 41

Azaisobutyronitrile (1 equiv.) and tributyltin hydride (1.3 equiv) were added to a solution of compound of Formula 40 (1 equiv.) in toluene and the reaction mixture was refluxed for about 14 hours. The reaction mixture was quenched by saturated solution of sodium bicarbonate and reaction mixture was extracted by ethyl acetate and washed with water followed by brine, dried over anhydrous sodium sulphate and concentrated under reduced pressure to afford the title compound.

### Example 26

### Preparation of Compound of Formula 42

Tetramethyl guanidine (2.2 equiv.) was added to a solution of compound of Formula 41 (1 equiv.) in dimethylformamide and the reaction mixture was heated at about 80-90° C. with stirring for about 8 hours. The reaction mixture was cooled to an ambient temperature, water was added to the reaction mixture and extracted with ethyl acetate. The organic layer was washed with water, brine, dried over anhydrous sodium sulphate, and concentrated under reduced pressure to yield the title compound.

### Example 27

### Preparation of Compound of Formula 43

N,N′-carbonyldiimidazole (3 equiv.) was added to a solution of compound of Formula 42 (1 equiv.) in a mixture of dimethylformamide and tetrahydrofuran (3:2) at an ambient temperature. The reaction mixture was cooled to 0° C. Sodium hydride (3 equiv.) was added to it in portion wise and reaction mixture was stirred for about 30 minutes. The reaction mixture was quenched by water, extracted with ethyl acetate, washed with water, brine, dried over anhydrous sodium sulphate, and concentrated under reduced pressure to afford the title compound.

### Example 28

### Preparation of Compound of Formula 44

Compound of Formula 43 (1 equiv.) and R¹¹(CH₂)₄NH₂ (2 equiv.) were taken in 10% water in acetonitrile and heated at about 70° C., stirred for about 14 hours. The reaction mixture was cooled to an ambient temperature and acetonitrile was removed under reduced pressure. The residue was taken in ethyl acetate and washed with water, brine, dried over anhydrous sodium sulphate, and filtered. The filtrate was concentrated under reduced pressure to form the crude product. The crude product was purified by silica gel column chromatography (thoroughly neutralized with triethylamine) using 25-30% acetone in hexane to afford the title compound.

### Example 29

### Preparation of Compound of Formula 45

Compound of Formula 44 was taken in methanol and refluxed for about 12 hours. The reaction mixture was cooled to an ambient temperature and methanol was evaporated under reduced pressure to form the crude product. The crude product was purified by silica gel column chromatography using 2-10% methanol in dichloromethane to afford the title compound.

The following compound was prepared following the procedure described in Example 24 to 29:
Compound No. 13: 11,12-dideoxy-3-O-decladinosyl-3-deoxo-6-O-methyl-3-oxo-12,11-[oxycarbonyl-(4-(4-(2-amino-pyrimidin-5-yl)-imidazol-1-yl)-butylimino)]erythromycin A

Mass (m/z) (M+1):815, m.pt. 117° C.

### Example 30

### Pharmacological Activity

Compounds disclosed herein displayed antibacterial activity in vitro especially against strains that are resistant to macrolides either due to efflux (mef strains) or ribosomal modification (erm) strains. These compounds are useful in treating community acquired pneumonia, upper and lower respiratory tract infections, skin and soft tissue infections, hospital acquired lung infections, bone and joint infections, and other bacterial infections, for example, mastitis, catheter infection, foreign body, prosthesis infections or peptic ulcer disease.

Minimum inhibitory concentration (MIC) has been an indicator of in vitro antibacterial activity widely used in the art.

### Procedure:

### Medium

a) Cation adjusted Mueller Hinton Agar (MHA-Difco)

### b) Trypticase Soya Agar (TSA)

### Inoculum Preparation

Cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well-isolated colonies were taken and saline suspensions were prepared in sterile densimat tubes. The turbidity of the culture was adjusted to 0.5-0.7 Mc Farland standard (1.5×10⁸ CFU/mL). The cultures were diluted 10 fold in saline to obtain inoculum sizes of approximately 1-2×10⁷ organisms/mL.

### Preparation of Drug Concentration

1 mg/mL concentration of stock solution of drugs was prepared in dimethylsulfoxide/distilled water/solvent given in National Committee for Clinical Laboratory Standards (NCCLS) manual. Serial two-fold dilutions of the compounds and standard drugs were prepared as per NCCLS manual.

The stock solution was changed according to the need of the experiment.

### Preparation of Agar Plates

Two mL of respective drug concentration was added to 18 mL of Molten Mueller Hinton agar to achieve the required range, for example 0.015 μg/mL-16 μg/mL. For fastidious cultures 1 mL of sheep blood was added in Molten Mueller Hinton agar.

MHA and MHA with 5% sheep blood plates without antibiotic for each set were prepared for controls. One MHA and MHA with 5% sheep blood plate without antibiotic for determining quality check for media was prepared.

### Preparation of Teflon Template

1 μL of each culture on each plate was replicated with the help of a replicator (i.e., Denley's multipoint replicator). The spots were allowed to dry and the plates were incubated for about 18-24 hours at 37° C. Fastidious cultures were incubated at 37° C. in a CO₂ incubator. The results were noted comparing with the control plates.

### Endpoint Definition

The concentration of drug at which there was complete disappearance of growth spot or formation of less than 10 colonies per spot was considered as Minimum Inhibitory Concentration (MIC).

The MICs of Quality Control (QC) strains were plotted on the QC chart for agar dilution method. If the MICs were within the range, the results interpreted by comparing MICs of standards against all organisms with those of test compounds.

### Precautions & Quality Control Measures

### Quality Control Strains

Staphylococcus aureus ATCC 29213
Enterococcus faecalis ATCC 29212
Eschericia coli ATCC 25922
Pseudomonas aeruginosa ATCC 27853
All 60 cultures were visually checked for purity.

Media Control: NCCLS disc diffusion assay using 10 μg discs of Gentamicin (Difco) against Pseudomonas aeruginosa ATCC 27853. A zone diameter of 16-21 mm was considered for optimum cation (Magnesium and Calcium) content of the media. The diameter was plotted in the media QC chart.

### REFERENCES

National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000).
National Committee for Clinical Laboratory Standards, Performance Standards for Antimicrobial Susceptibility Testing—Twelfth informational supplement, M 100-12, Vol. 22 No. 1 (January 2002).

Compounds of this invention have shown good activity against microbial strains, for example, Staphylococcus aureus, Streptococcus pneumoniae, Haemophilus influenzae, Moraxella catarrhalis, Streptococcus pyogenes, enterococci species, Helicobacter pylori, E. faecalis or combination thereof.

Compounds of this invention have shown good activity against microbial strains, for example, Staphylococcus aureus, Streptococcus pneumoniae, Haemophilus influenzae, Moraxella catarrhalis, Streptococcus pyogenes, Enterococcus faecium, Helicobacter pylori and E. faecalis
a) The compounds disclosed herein exhibited MIC values against Staphylococcus aureus in the range between about 0.5 μg/mL to 4 μg/mL.
b) The compounds disclosed herein exhibited MIC values against sensitive Streptococcus pneumoniae in the range between about 0.03 μg/mL to about >16 μg/mL.
c) The compounds disclosed herein exhibited MIC values against resistant Streptococcus pneumoniae in the range between about 0.03 μg/mL to about >16 μg/mL
d) The compounds disclosed herein exhibited MIC values against Haemophilus influenzae in the range between about 0.06 μg/mL to about >16 μg/mL.
e) The compounds disclosed herein exhibited MIC values against Moraxella species in the range between about 0.06 μg/mL to about 4 μg/mL
f) The compounds disclosed herein exhibited MIC values against sensitive Streptococcus pyogenes in the range between about 0.03 μg/mL to about 0.125 μg/mL.
g) The compounds disclosed herein exhibited MIC values against resistant Streptococcus pyogenes in the range between about 0.03 μg/mL to about >16 μg/mL.
h) The compounds disclosed herein exhibited MIC values against MRSA about >16 μg/mL
i) The compounds disclosed herein exhibited MIC values against sensitive enterococci species in the range between about 0.06 μg/mL to about >16 μg/mL

### Example 31

### Pharmacological Testing

### Acne Vulgaris

Minimum inhibitory concentration (MIC) of compound described herein and standard drugs are determined against five isolates of Propionibacterium acnes [Propionibacterium acnes I (Sensitive), Propionibacterium acnes II (Sensitive), Propionibacterium acnes 6523, and two isolates of Propionibacterium acnes (Telithromycin Resistant)] as per NCCLS guidelines (M11-A5) by agar dilution method. Brucella agar (Difco) with hemin (5 μg/ml) and vitamin K1 (1 μg/ml) supplemented with 5% (v/v) laked sheep blood is used as media and all plates are incubated at 37° C. in anaerobic jar under anaerobic condition created by using Anaxomat.

The compounds disclosed herein exhibited MIC values against Propionibacterium acnes in the range between about 0.03 μg/mL to about 0.25 μg/mL

1. A compound having the structure of Formula I, 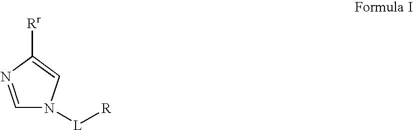
pharmaceutically acceptable salts, pharmaceutically acceptable solvates, enantiomers, diastereomers or polymorphs thereof, wherein:
R^{r} is 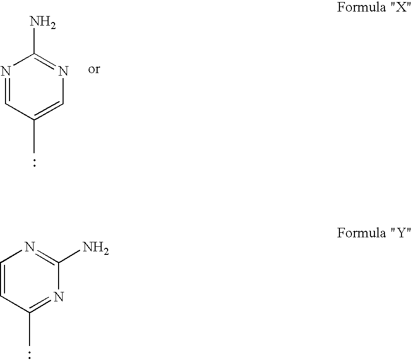
NH₂ is optionally substituted;
substitutents are alkyl or form a heterocyclic ring together with nitrogen atom;
L is a linker;
R is 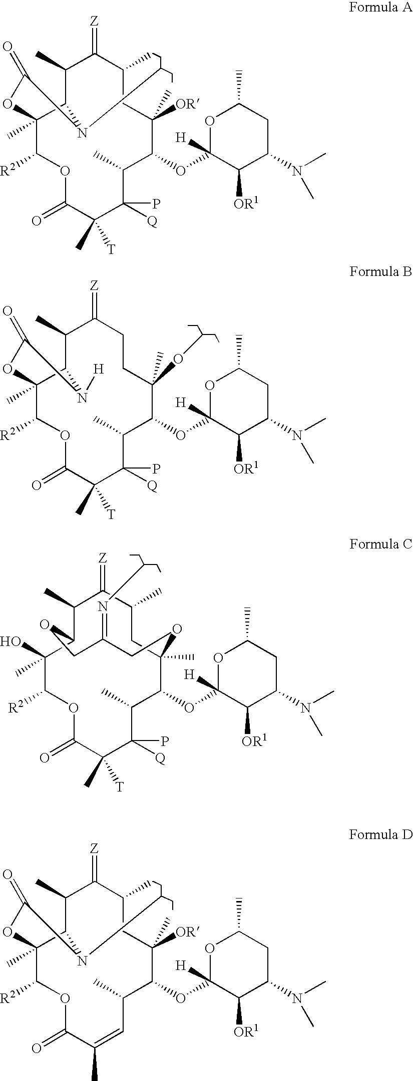
R¹ is hydrogen or hydroxy protecting group;
R² is alkyl, alkenyl or alkynyl;
R′ is alkyl or —(CH₂)ᵣ—U;
r is an integer of from 1 to 4;
U is alkenyl or alkynyl;
Z is oxygen, sulfur or NAc, NOR⁴;
R⁴ is hydrogen, alkyl or arylalkyl;
T is hydrogen, halogen, cyano or alkyl;
P and Q are independently hydrogen, hydroxy, OC(=Z′)VR³ or 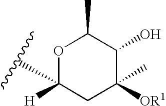
P and Q together form oxo or thioxo group;
Z′ is oxygen or sulfur;
V is —W(CH₂)ₖ—;
k is an integer of from 1 to 6;
W is no atom, —NR⁵— or oxygen;
R⁵ is hydrogen or alkyl;
alkylene chain of —W(CH₂)ₖ— is optionally substituted with alkyl, hydroxy or alkoxy;
R³ is alkyl, aryl or heterocycle.

2. The compound of claim 1, wherein linker is (CH₂)₄.

3. A compound according to claim 1 and 2, which is selected from a group consisting of:
R^{r} is Formula “X”; L is (CH₂)₄; R is Formula A [P and Q together form oxo group, R¹=H, R²=Et, Z=O, R′=Me, T=H],
R^{r} is Formula “Y”; L is (CH₂)₄; R is Formula A [P and Q together form oxo group, R¹=H, R²=Et, Z=O, R′=Me, T=H],
R^{r} is Formula “X”; L is (CH₂)₄; R is Formula A [P═H, Q=O-(2-pyridyl acetyl), R¹=H, R²=Et, Z=O, R′=Me, T=H],
R^{r} is Formula “X”; L is (CH₂)₄; R is Formula A [P═H, Q=O-(3-pyridyl acetyl), R¹=H, R²=Et, Z=O, R′=Me, T=H],
R^{r} is Formula “X”; L is (CH₂)₄; R is Formula A [P═H, Q=O-(2-fluorophenyl acetyl), R¹=H, R²=Et, Z=O, R′=Me, T=H],
R^{r} is Formula “X”; L is (CH₂)₄; R is Formula A [P═H, Q=O-(3-fluorophenyl acetyl), R¹═H, R=Et, Z=O, R′=Me, T=H],
R^{r} is Formula “Y”; L is (CH₂)₄; R is Formula A [P═H, Q=O-(3-fluorophenyl acetyl), R¹=H, R¹=Et, Z=O, R′=Me, T=H],
R^{r} is Formula “Y”; L is (CH₂)₄; R is Formula A [P═H, Q=O-(2-fluorophenyl acetyl), R¹=H, R²=Et, Z=O, R′=Me, T=H],
R^{r} is Formula “Y”; L is (CH₂)₄; R is Formula A [P═H, Q=O-(3-pyridyl acetyl), R¹=H, R²=Et, Z=O, R′=Me, T=H],
R^{r} is Formula “Y”; L is (CH₂)₄; R is Formula A [P═H, Q=O-(2-pyridyl acetyl), R¹=H, R²=Et, Z=O, R′=Me, T=H],
R^{r} is Formula “X”; L is (CH₂)₄; R is Formula A [P and Q together form oxo group, R¹=H, R²=Et, Z=O, R′=Me, T=F],
R^{r} is Formula “X”; L is (CH₂)₄; R is Formula A [P═H, Q=H, R¹═H, R²=Et, Z=O, R′=Me, T=H],
R^{r} is Formula “X”; L is (CH₂)₄; R is Formula D [R¹=H, R²=Et, Z=O, R′=Me]

4. A pharmaceutical composition comprising therapeutically effective amounts of one or more compounds of claim 1 together with one or more pharmaceutically acceptable carrier, excipients, diluents or mixture thereof.

5. A pharmaceutical composition of claim 4 in combination with one or more therapeutic agents which can be used for treating skin conditions, selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, or azelaic acid.

6. A method for treating or preventing a condition caused by or contributed to by bacterial infection, comprising administering to a mammal in need thereof therapeutically effective amounts of one or more compounds of claim 1.

7. The method according to claim 6, wherein the said condition is selected from community acquired pneumonia, upper and lower respiratory tract infections, skin and soft tissue infections, acne vulgaris, hospital acquired lung infections, hospital acquired bone and bone joint infections, mastitis, catheter infection, foreign body, prosthesis infections or peptic ulcer disease.

8. The method according to claim 6, wherein the bacterial infection is caused by gram positive, gram negative or anaerobic bacteria selected from Staphylococci, Streptococci, Enterococci, Haemophilus, Moraxalla spp., Chlamydia spp., Mycoplasm, Legionella spp., Mycobacterium, Helicobacter, Clostridium, Propionibacterium, Bacteroides, Corynebacterium or Enterobactericeae.

9. A compound selected from:

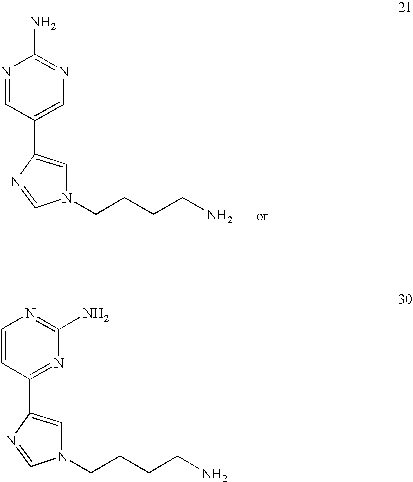

10. A process for preparing a compound of Formula 21 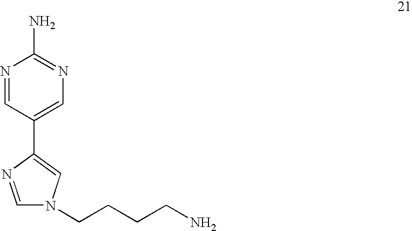
comprising the steps of:
(a) coupling a compound of Formula 14 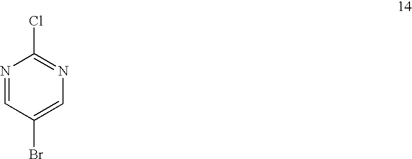
with a compound of Formula 15 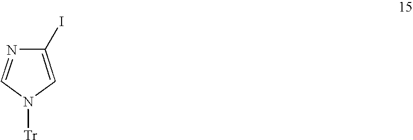
to give a compound of Formula 16 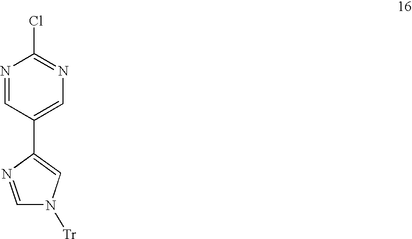
wherein Tr is trityl
(b) deprotecting a compound of Formula 16 to give a compound of Formula 17 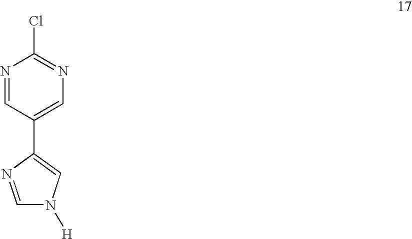
(c) reacting a compound of Formula 17 with N-(4-bromobutyl)-phthalimide to give a compound of Formula 18 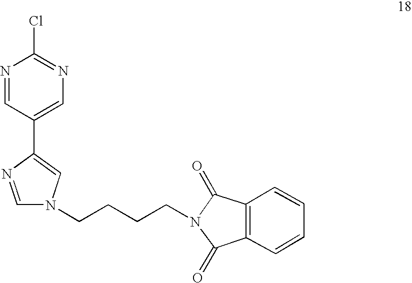
(d) reacting a compound of Formula 18 with alkali metal azide to give a compound of Formula 19 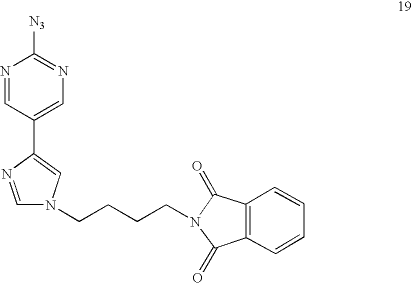
(e) reducing a compound of Formula 19 to give a compound of Formula 20 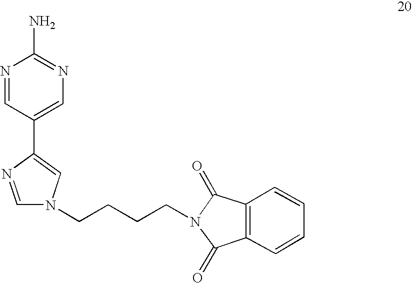
(f) reacting a compound of Formula 20 with hydrazine monohydrate to give a compound of Formula 21.

11. A process for preparing a compound of Formula 30 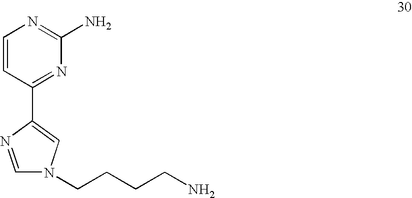
comprising the steps of:
(a) reacting a compound of Formula 22 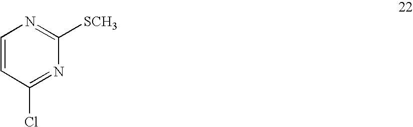
with hydroiodic acid to give a compound of formula 23 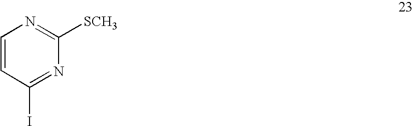
(b) coupling a compound of Formula 23 with a compound of Formula 15 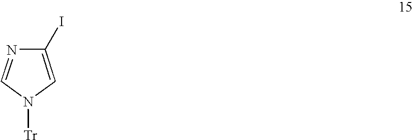
to give a compound of Formula 24 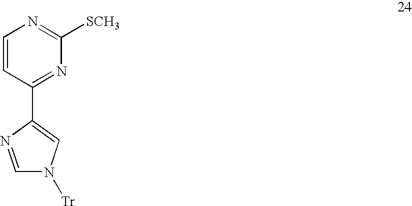
wherein Tr is trityl
(c) deprotecting a compound of Formula 24 to give a compound of Formula 25 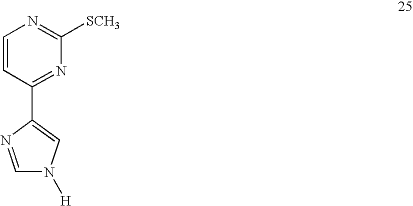
(d) reacting a compound of Formula 25 with N-(4-bromobutyl)-phthalimide to give a compound of Formula 26 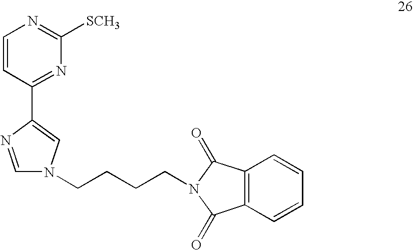
(e) oxidizing a compound of Formula 26 to give a compound of Formula 27 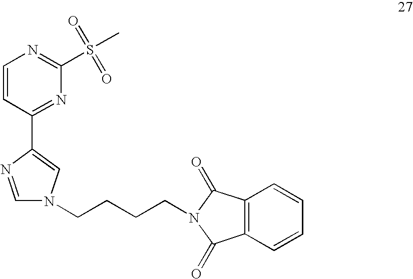
(f) reacting a compound of Formula 27 with alkali metal azide to give a compound of Formula 28 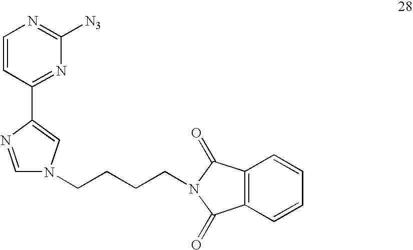
(g) reducing a compound of Formula 28 to give a compound of Formula 29 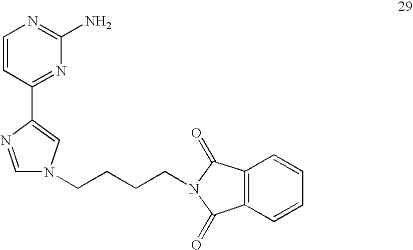
(h) reacting a compound of Formula 29 with hydrazine monohydrate to give a compound of Formula 30.

12. A process for preparing a compound of Formula 9 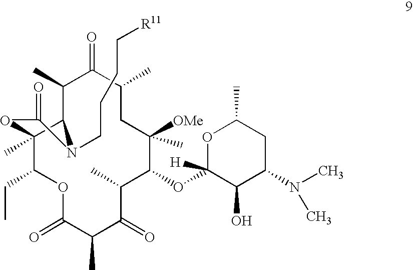
comprising the steps of:
(a) reacting a compound of Formula 7 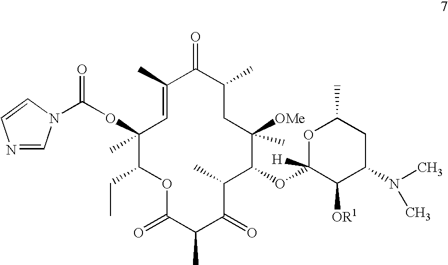
with a compound of formula R¹¹(CH₂)₄NH₂ to give a compound of Formula 8. 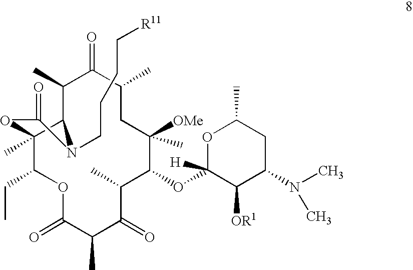
(b) deprotecting a compound of Formula 8 to give a compound of Formula 9 wherein R¹¹ is 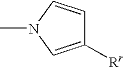
R^{r} is 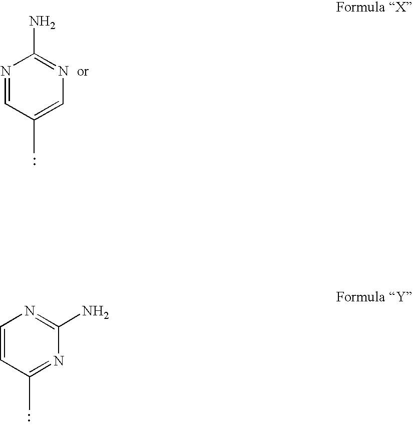
NH₂ is optionally substituted;
substitutents are alkyl or form a heterocyclic ring together with nitrogen atom;
R¹ is hydrogen or hydroxy protecting group.

13. A process for preparing a compound of Formula 13 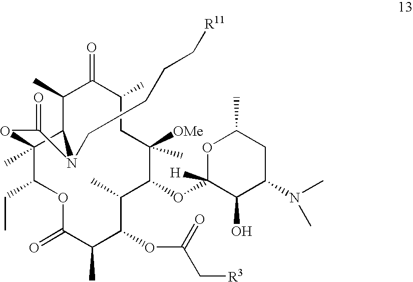
comprising the steps of:
(a) reacting a compound of Formula 11 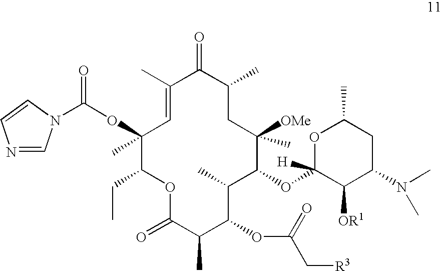
with a compound of formula R¹¹(CH₂)₄NH₂ to give a compound of Formula 12. 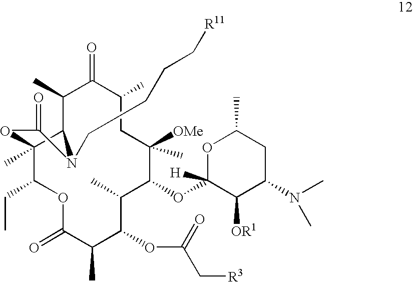
(b) deprotecting a compound of Formula 12 to give a compound of Formula 13. wherein R¹¹ is 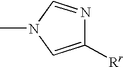
R^{r} is 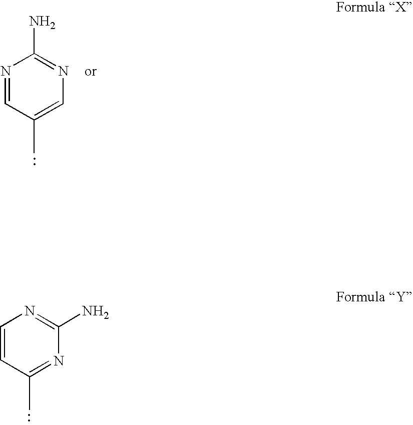
NH₂ is optionally substituted;
substitutents are alkyl or form a heterocyclic ring together with nitrogen atom;
R¹ is hydrogen or hydroxy protecting group;
R³ is alkyl, aryl or heterocycle.

14. A process for preparing a compound of Formula 34 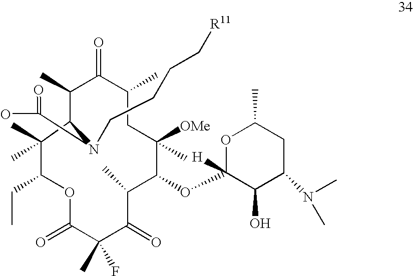
comprising the steps of:
(a) reacting a compound of Formula 32 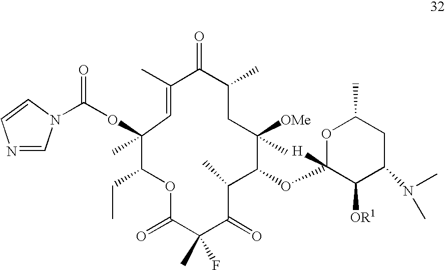
with a compound of formula R¹¹(CH₂)₄NH₂ to give a compound of Formula 33. 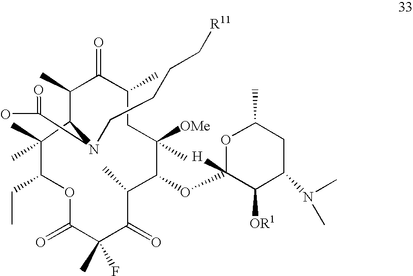
(b) deprotecting a compound of Formula 33 to give a compound of Formula 34, wherein R¹¹ is 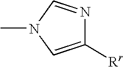
R^{r} can be 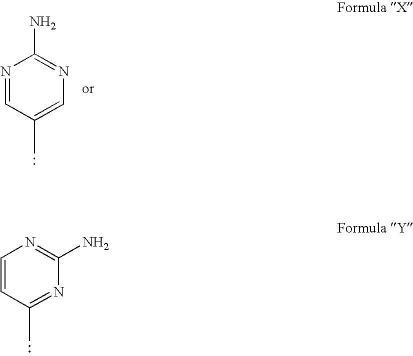
NH₂ can be optionally substituted;
substitutents can be alkyl or can form a heterocyclic ring together with nitrogen atom;
R¹ can be hydrogen or hydroxy protecting group.

15. A process for preparing a compound of Formula 39 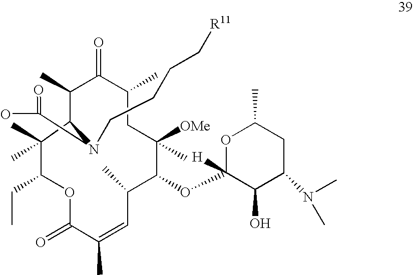
comprising the steps of:
(a) reacting a compound of Formula 37 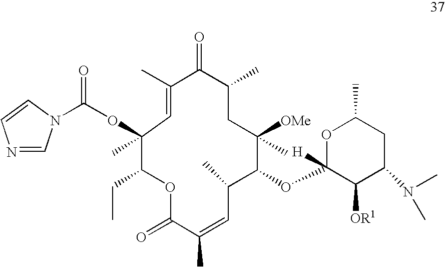
with a compound of formula R¹¹(CH₂)₄NH₂ to give a compound of Formula 38. 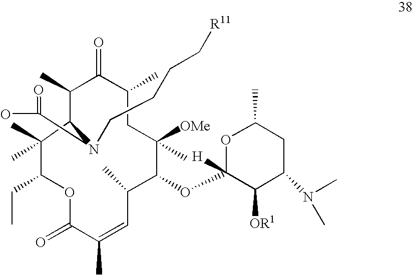
(b) deprotecting a compound of Formula 38 to give a compound of Formula 39. wherein R¹¹ is 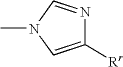
R^{r} is 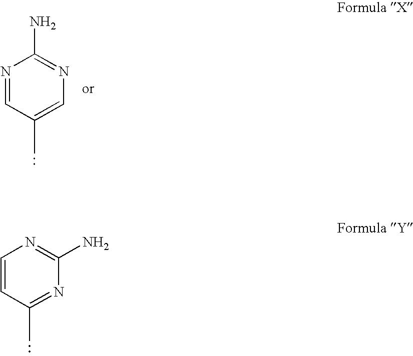
NH₂ is optionally substituted;
substitutents are alkyl or form a heterocyclic ring together with nitrogen atom;
R¹ is hydrogen or hydroxy protecting group.

16. A process for preparing a compound of Formula 45 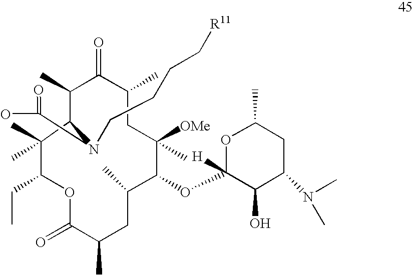
(a) reacting a compound of Formula 43 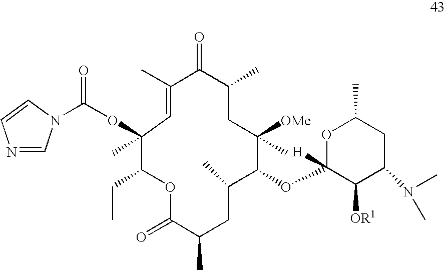
with a compound of formula R¹¹(CH₂)₄NH₂ to give a compound of Formula 44. 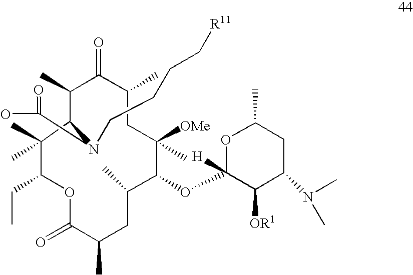
(b) deprotecting a compound of Formula 44 to give a compound of Formula 45. wherein R¹¹ is 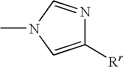
R^{r} is 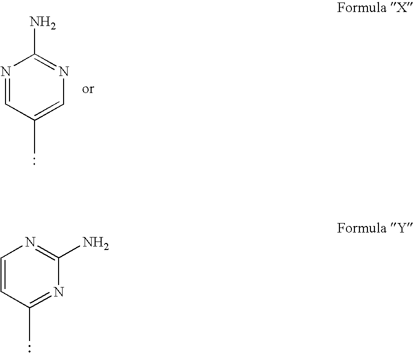
NH₂ is optionally substituted;
substitutents are alkyl or form a heterocyclic ring together with nitrogen atom;
R¹ is hydrogen or hydroxy protecting group.
